(19) United States
(12) Patent Application Publication
(10) Pub. No.: US 2025/0161453 A1
(43) Pub. Date: May 22, 2025

(54) LIGHT-ACTIVATABLE, SUSTAINED-EXPOSURE ETHANOL INJECTION

(71) Applicant: University of Maryland, College Park, College Park, MD (US)

(72) Inventors: Huang Chiao Huang; Jenna Mueller, University Park, MD (US); Jeffrey Yang, Laurel, MD (US); Chen-Hua Ma, College Park, MD (US)

(21) Appl. No.: 18/839,523

(22) PCT Filed: Mar. 10, 2023
(86) PCT No.: PCT/US2023/064137

§ 371 Date: Aug. 19, 2024

Related U.S. Application Data
(60) Provisional application No. 63/269,238, filed on Mar. 11, 2022.

(51) Int. Cl. A61K 41/00; A61K 31/337; A61K 31/704; A61K 31/7088; A61K 33/243; A61K 38/19; A61K 47/10; A61K 47/38; A61K 48/00; A61P 35/00

(52) U.S. Cl. CPC A61K 41/0071; A61K 31/337; A61K 31/704; A61K 31/7088; A61K 33/243; A61K 38/19; A61K 41/0061; A61K 47/10; A61K 47/38; A61K 48/0033; A61P 35/00

(57) ABSTRACT

Described herein are light-activatable, sustained-exposure compositions for the treatment and monitoring of diseases along with methods of making and using the same, wherein the compositions comprise a solvent, a cellulosic polymer, a photosensitizer, and optionally additional therapeutic agents. In some embodiments, an additional therapeutic agent is included. The compositions can be administered to a target in need, such as a tumor or lesion, and activated with a light source to induce necrosis or apoptosis and to track the distribution of the composition at the target site. Beneficially the compositions provide synergistic ablation and photodynamic efficacy.

### CROSS-REFERENCE TO RELATED APPLICATIONS

This application claims priority to U.S. Provisional Application No. 63/269,238, filed Mar. 11, 2022, the disclosure of which is hereby incorporated by reference in its entirety.

### TECHNICAL FIELD

The present disclosure generally relates to methods, systems, devices, and compositions for the delivery and visualization of materials such as drugs or other therapeutics, along with the use of the same for the treatment of disorders or conditions in a subject.

### TECHNICAL BACKGROUND

Local drug injection into malignancies and lesions is a promising strategy to enhance treatment efficacy and decrease systemic toxicity in patients. For example, intratumoral drug injections have a particular appeal for patients with locally advanced, unresectable tumors. Direct injection of therapeutic agents into tumors may also allow for higher local drug concentrations with low systemic toxicities, compared to systemic administration routes (e.g., intravenous, intraperitoneal, oral). While local drug administration possesses certain advantages over systemic injection routes, challenges remain that limit the effectiveness of local drug injection for disease management. For example, the heterogeneous distribution of injectate in tumors, especially in the presence of intratumoral septa, can prevent full eradication of the tumor volume. Furthermore, crack formations from injecting into non-capsulated tumors can lead to drug or therapeutic leakage out of tumors, damaging nearby normal tissues and decreasing treatment efficacy.

Current approaches for improving local drug retention in tissues include modifying injection parameters, for example by modifying needle design and injection rate to generate an appropriate distribution, as well as tuning the physicochemical properties of the drug formulation to encourage retention at the injection site. However, even where such approaches provide incremental improvements in local drug retention, a key component of successful treatment is the ability to monitor drug distribution over time. Combining optical contrast agents with drugs is one of the strategies to monitor local drug delivery in both ex vivo and in vivo contexts. However, incorporation of optical contrast agents into a targeted therapeutic compound is challenging due to a number of issues such as poor sensitivity, poor quantitative characterization of the injectates, background signaling (e.g., specular reflection, autofluorescence), compatibility with the therapeutic composition, and toxicity to non-target cells. These issues are key in obtaining quantitative biodistribution readings and optimizing local drug delivery protocols to achieve sufficient drug coverage while minimizing drug leakage to normal tissues.

Furthermore, combination therapy is increasingly a cornerstone treatment modality for many diseases and conditions. However, the administration of combination therapies frequently involves the separate administration of two or more drugs or therapeutic agents. Individual administration is sometimes necessitated by differences in dosing, suitable administration method, and composition compatibility (e.g., undesirable interactions or instability that would result from combining two drugs into a single formulation).

Thus, overall, there remains a need to develop formulations that combine two or more therapeutic agents or drugs, the administration of which can be highly localized and easily monitored, regardless of the site of administration.

More particularly, there is a need for facile and sensitive methods to improve both the retention and monitoring of injectates comprising one or more drugs or therapeutic agents.

There is a need to use optical contrast agents, such as photosensitizers with fluorescent properties, to evaluate the drug distribution and efficacy of therapeutics and drugs.

There is a further need to provide stable, compatible compositions that enable the use of multiple active agents and result in superior or synergistic efficacy in treating a disease or condition.

These and other objects, advantages, and features of the present disclosure will become apparent from the following specification taken in conjunction with the claims set forth herein.

### BRIEF SUMMARY

The present disclosure relates to systems, devices, and methods for material delivery and visualization of material distribution. In certain embodiments, the materials being delivered and visualized may be drugs or other therapeutics. In certain aspects, the systems, devices, and methods may be used for percutaneous injection, for example, percutaneous ethanol injection (PEI). In certain aspects, the system device and method may include a stable, multi-agent formulation, which may be administered via percutaneous injection. In certain aspects, the formulation includes, alcohol, for example, ethanol; a polymer, for example, ethyl cellulose (EC); and a photosensitizer, which may be a benzoporphyrin derivate. In certain aspects, the present disclosure relates to methods of making the formulation, which may include dissolving the polymer in the alcohol and dissolving the photosensitizer in the polymer-alcohol solution. In an example, the final formulation contains approximately 6% w/v of EC dissolved in alcohol and a concentration of photosensitizer approximately 20 μM. In certain aspects, the system, device, and methods may include a light source, for example, a non-ionizing electromagnetic radiation source producing near-infrared light, to activate the photosensitizers in the formulation. In certain aspects, this produces cytotoxic reactive oxygen species for photodynamic therapies. In certain aspects, the activated (or excited) photosensitizers may generate fluorescence signals for image-guided drug delivery, light dosimetry, and visualizations of the material delivery.

The present disclosure relates to a light-activatable, sustained-exposure composition for treating a variety of diseases, including cancer. The composition comprises a solvent comprising an alcohol, a cellulosic polymer, and a photosensitizer. The solvent may include phenol, methanol, ethanol, absolute alcohol, isopropanol, propanol, butanol, isobutanol, glycerol, propyl iodide, lipiodol, glycerol, polidocanol, or a combination thereof. The cellulosic polymer may include ethyl cellulose (EC), methyl ethyl cellulose (MEC), carboxymethyl cellulose (CMC), carboxymethyl ethyl cellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), ethyl hydroxyethyl cellulose (EHEC), cyanothyl cellulose, alginate, chitosan, or a combination thereof. The photosensitizer may include a benzoporphyrin derivative, HPPH, WST-11, ALA, methyl-ALA, hexyl-ALA, chlorin e6 trisodium, mono-L-aspartyl chlorin e6, talaporfin sodium, redaporfin, temoporfin foscan, photofrin, phthalocyanine, or a combination thereof. In an embodiment, the composition may also include an additional therapeutic agent, such as a chemotherapeutic agent, an immunotherapeutic agent, a gene therapy agent, or a combination thereof. In an embodiment, the photosensitizer is present in an amount of between about 5 μM to about 100 μM, while the cellulosic polymer is present in an amount of 1-15% (w/v) relative to the total weight of the composition and the solvent is present in an amount of 1-99% (v/v) relative to the total volume of the composition. In an embodiment, the composition may comprise between about 1% (w/v) and 15% (w/v) cellulosic polymer-solvent.

In another aspect of the disclosure, a method of treating a disease is provided. The method includes administering to a target in need a therapeutically effective amount of the light-activatable, sustained-exposure composition and exposing the target to a light source to activate the photosensitizer. In an embodiment, the administering may occur by injection and may take place between once a week to once a month over a period of three months to a year. The composition may become a gel upon administering and may be used to treat tumors or lesions. In an embodiment, the disease may include liver cancer, pancreatic cancer, cervical cancer, or breast cancer. The method may also include monitoring the distribution of the composition in the target, ablating a portion or all of the target, and generating reactive oxygen species in an amount sufficient to induce necrosis or apoptosis in a portion or all of the target.

In an embodiment, the methods involve administering a liquid composition that can become a gel upon administering. The administration can be done by injection and can occur between once a day to once a month over a period of three months to a year.

In an embodiment, the administering occurs at an injection rate of between about 10 mL/hour to about 100 mL/hour. In an embodiment, the administering occurs at an injection volume of between about 100 μL to about 5000 μL. In an embodiment, the administering occurs at an insertion depth of between about 4 mm to about 120 mm. In an embodiment, the light source reaches up to 1.5 cm in the target.

In an embodiment, the target of the treatment is a tumor or lesion, and the diseases that can be treated using this method include liver cancer, pancreatic cancer, cervical cancer, head and neck cancer, skin cancer, oral cancer, and breast cancer. In particular, this disclosure provides an efficient and effective method for administering cancer treatment over a period of time, with a minimized frequency of administration, and targeted delivery to the tumor or lesion site.

While multiple embodiments are disclosed, still other embodiments of the present disclosure will become apparent based on the detailed description, which shows and describes illustrative embodiments of the disclosure. The foregoing summary is illustrative only and is not intended to be in any way limiting. In addition to the illustrative aspects, embodiments, and features described above, further aspects, embodiments, and features of the present technology are apparent from the following drawings and the detailed description, which shows and describes illustrative embodiments of the present technology.

Each feature of the technology described herein may be combined with any one or more other features of the disclosure, e.g., the methods may be used with any composition described herein. For example, the disclosure encompasses embodiments wherein the elements of each claim may be combined with the elements of any one or more of the other claims. Accordingly, the drawings and detailed description are to be regarded as illustrative and not restrictive.

### BRIEF DESCRIPTION OF THE DRAWINGS

FIG. 1A shows an example injection setup for obtaining a distribution volume for analysis. In particular, the benzoporphyrin derivative-ethyl cellulose-ethanol (BPD-EC-Ethanol) solution is injected into a tissue-mimicking phantom via an automated syringe pump. Any pooled retrograde backflow leakage (outlined, yellow arrow) can be removed and measured.

FIG. 1B shows a frontal cross-section of the BPD-EC-Ethanol distribution in the phantom. Scale bars represent 5 mm.

FIG. 1C depicts a side cross-section of the BPD-EC-Ethanol distribution in the phantom. Scale bars represent 5 mm.

FIG. 1D shows how punch biopsies of the distribution from FIG. 1B are inserted into a transparent, 96-well flat-bottom microplate.

FIG. 2A shows a representative fluorescence image of the BPD-EC-Ethanol distribution in tumor-mimicking phantoms, in particular a front view in the phantom. The scale bar represents 5 mm.

FIG. 2B is a front-view binary image for the phantom to trace the distribution in the phantom. The scale bar represents 5 mm.

FIG. 2C shows a representative fluorescence image of the BPD-EC-Ethanol distribution in tumor-mimicking phantoms, in particular a side view in the phantom. The scale bar represents 5 mm.

FIG. 2D is a side-view binary image for the phantom to trace the distribution in the phantom. The scale bar represents 5 mm.

FIG. 3A is a representative digital image of the BPD-EC-Ethanol distribution in swine liver tissue. The arrow indicates the injection site. The scale bar represents 5 mm.

FIG. 3B is a representative fluorescence image of the BPD-EC-Ethanol distribution in swine liver tissue. The arrow indicates the injection site. The scale bar represents 5 mm.

FIG. 3C is a representative front view binary image of the BPD-EC-Ethanol distribution in swine liver tissue. The arrow indicates the injection site. The scale bar represents 5 mm.

FIG. 3D is a representative digital image of the BPD-EC-Ethanol distribution in swine liver tissue. The arrow indicates the injection site. The scale bar represents 5 mm.

FIG. 3E is a representative fluorescence image of the BPD-EC-Ethanol distribution in swine liver tissue. The arrow indicates the injection site. The scale bar represents 5 mm.

FIG. 3F is a representative side view binary image of the BPD-EC-Ethanol distribution in swine liver tissue. The arrow indicates the injection site. The scale bar represents 5 mm.

FIG. 4 comprises representative fluorescence and threshold images of the injection of light-activatable, sustained-exposure ethanol injection technology (LASEIT) comprising BPD-EC-ethanol and controls (BPD-ethanol, BPD in phosphate buffered saline (PBS)) in swine liver tissue. The images presented are a cross section through the depot.

FIG. 5 is a chart of the average distribution volumes of the swine liver depots of FIG. 4, demonstrating that LASEIT significantly increases depot volume in tissues compared to the controls.

FIG. 6A shows a schematic diagram of a soluble mixture of the photosensitizer drug benzoporphyrin derivative (BPD), ethyl cellulose (EC), and ethanol (EtOH) that can form a gel depot (BPD-EC-EtOH) in water.

FIG. 6B shows representative images of a soluble mixture of the photosensitizer drug benzoporphyrin derivative (BPD), ethyl cellulose (EC), and ethanol (EtOH) that can form a gel depot (BPD-EC-EtOH) in water.

FIG. 6C shows a schematic diagram of the BPD-EC-EtOH gel depot in agar-based tissue-mimicking phantoms.

FIG. 6D shows the formation of the BPD-EC-EtOH gel depot in agar-based tissue-mimicking phantoms (scale bar=5 mm).

FIG. 6E is a chart showing that, upon red light (690 nm) activation of BPD-EC-EtOH, the fluorescence signal generated from BPD can be used for imaging of the depot.

FIG. 7A depicts representative images showing BPD-EC-EtOH gel depot formation in swine liver tissue.

FIG. 7B is a chart showing the fluorescence signal of BPD-EC-EtOH in swine liver tissue.

FIG. 8A shows the light propagation of three samples: the EC-based gel depot, water, and a light scattering agent (0.1% Intralipid®).

FIG. 8B is a photograph showing measurements of light propagation in pipette.

FIG. 8C is a chart depicting measurements of light propagation in pipette.

FIG. 8D is a diagram showing how liver tissues injected with BPD-EC-EtOH, BPD-EtOH, or BPD-PBS were irradiated, and fluorescence spectra detected.

FIG. 8E is a series of graphs measuring fluorescence and wavelength for the three samples (BPD-EC-EtOH, BPD-EtOH, or BPD-PBS).

FIG. 8F is a chart measurement of fluorescence versus distance (mm) for the three samples (BPD-EC-EtOH, BPD-EtOH, BPD-PBS).

FIG. 9A is a diagram showing the process for evaluating the synergistic effects of BPD-based photodynamic therapy in combination with ethanol ablation in liver and pancreatic cancer cells.

FIG. 9B comprises two graphs showing cell viability of liver and pancreatic cancer cells following PDT and ablation treatment.

FIG. 9C is a combination index evaluating the degree of drug interaction between treatments involving PDT and ablation.

FIG. 10A comprises IVIS imaging of mouse pancreatic tumors given treatments of BPD-EC-EtOH, BPD alone, and no treatment.

FIG. 10B is a chart showing fold changes in the tumor as a function of time for the mouse treatment groups.

FIG. 10C is a chart showing tumor growth rates for the mouse treatment groups.

FIG. 11A shows images of light-activatable, sustained-exposure ethanol injection technology (LASEIT) volume distribution in tissue surrogates.

FIG. 11B is a chart showing the distribution area of the injection.

FIG. 11C is a chart showing the solidity post-injection.

FIG. 11D is a chart showing the circularity post-injection.

FIG. 12A shows a panel of representative fluorescence and digital images of LASEIT injections as a function of infusion volume.

FIG. 12B shows LASEIT distribution area as a function of infusion volume (n=5), where all error bars are SEM. (**: p<0.01).

FIG. 12C shows a panel of representative fluorescence and digital images of LASEIT injections as a function of infusion rate.

FIG. 12D shows LASEIT distribution area as a function of infusion rate (n=5), where all error bars are SEM (*: p<0.05).

FIG. 13A is a schematic diagram of a process for evaluating LASEIT distribution volume in excised swine liver.

FIG. 13B shows representative digital fluorescence, and segmented binary images of both LASEIT (top row) and control (bottom row) in excited swine liver.

FIG. 13C is a chart evaluating the distribution volume for BPD-EtOH and LASEIT.

FIG. 14A contains representative digital images of unstained, NADH-diaphorase, and H&E tumor slides treated with LASEIT or its monotherapy counterparts, along with the tumor slides' representative fluorescence images, 7 days post-treatment.

FIG. 14B contains representative digital images of unstained, NADH-diaphorase, and H&E tumor slides treated with LASEIT or its monotherapy counterparts, along with the tumor slides' representative fluorescence images, 14 days post-treatment.

FIG. 14C is a chart showing MIA PaCa-2 tumor size monitoring after treatment with LASEIT or its monotherapy counterparts.

Various embodiments of the present disclosure will be described in detail regarding the drawings. Reference to various embodiments does not limit the scope of the disclosure. Figures represented herein are not limitations on the various embodiments according to the disclosure and are presented as an example illustration of the disclosure.

### DETAILED DESCRIPTION OF THE PREFERRED EMBODIMENTS

The embodiments of this disclosure are not limited to particular types of compositions or methods, which can vary. It is further to be understood that all terminology used herein is to describe particular embodiments only and is not intended to be limiting in any manner or scope. For example, as used in this specification and the appended claims, the singular forms “a,” “an” and “the” can include plural referents unless the context indicates otherwise. Unless indicated otherwise, “or” can mean any one alone or any combination thereof, e.g., “A, B, or C” means the same as any of A alone, B alone, C alone, “A and B,” “A and C,” “B and C” or “A, B, and C.” Further, all units, prefixes, and symbols may be denoted in their SI accepted form.

As used herein, the terms “comprise,” comprises, “comprising,” “include,” “includes,” and “including” can be interchanged and are to be construed as at least having the features to which they refer while not excluding any additional unspecified features.

Numeric ranges recited within the specification are inclusive of the numbers defining the range and include each integer within the defined range. Throughout this disclosure, various aspects of this disclosure are presented in range formats. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6, and decimals and fractions, for example, 1.2, 3.8, 1½, and 4¼ This applies regardless of the breadth of the range.

So that the present disclosure may be more readily understood, certain terms are first defined. Unless defined otherwise, all technical and scientific terms used herein have the same meaning as commonly understood by one skilled in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The terms “a,” “an.” and “the” include both singular and plural referents.

The term “or” is synonymous with “and/or” and means any one member or combination of members of a particular list.

The term “about,” as used herein, refers to variation in the numerical quantity that can occur, for example, through typical measuring techniques and equipment, with respect to any quantifiable variable, including, but not limited to, mass, volume, time, temperature, pH, reflectance, whiteness, etc. Further, in practical handling procedures, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

As referenced herein, the term “combination therapy” describes a method of treating a disease or condition using two or more drugs or therapeutic agents.

As used herein, the term “drug” is used to describe a chemical substance or composition that is intended for use in the diagnosis, treatment, prevention, or mitigation of disease or medical conditions in humans or animals. A drug may include small molecule compounds, biologics, peptides, proteins, antibodies, nucleic acids, or any other therapeutic agent that is capable of interacting with a biological system to produce a desired effect.

Similarly, “therapeutic” and “therapeutic agent” as used herein broadly refers to any substance having biological or chemical activity or composition thereof that is used to treat, prevent, mitigate, or diagnose a disease or medical condition in humans or animals. This may include drugs, as well as medical devices, diagnostic tools, surgical procedures, and other forms of therapy that are intended to improve the health or well-being of a patient. Therapeutic interventions may be directed at specific targets within the body, such as receptors, enzymes, or other biological pathways, and may be designed to produce a wide range of therapeutic effects, including symptom relief, disease prevention, or disease modification.

As used herein, a “photosensitizer” or “photoreactive agent” is a compound or composition that is useful in photodynamic therapy in that it absorbs electromagnetic radiation and emits energy sufficient to exert a therapeutic effect, e.g., the impairment or destruction of unwanted cells or tissue, or sufficient to be detected in diagnostic applications. Photodynamic therapy according to the disclosure can be performed using any of a number of photoactive compounds. For example, the photosensitizer can be any chemical compound that collects in one or more types of selected target tissues and, when exposed to the light of a particular wavelength, absorbs the light and induces impairment or destruction of the target tissues. Virtually any chemical compound that homes to a selected target and absorbs light may be used in examples of this disclosure. Preferably, the photosensitizer is nontoxic to the patient to which it is administered and is capable of being formulated in a nontoxic composition. The photosensitizer is also preferably nontoxic in its photodegraded form. Ideal photosensitizers are characterized by a lack of toxicity to cells in the absence of the photochemical effect and are readily cleared from non-target tissues.

The methods, systems, apparatuses, and compositions disclosed herein may comprise, consist essentially of, or consist of the components and ingredients described herein as well as other ingredients not described herein. As used herein, “consisting essentially of” means that the methods, systems, apparatuses, and compositions may include additional steps, components, or ingredients, but only if the additional steps, components, or ingredients do not materially alter the basic and novel characteristics of the claimed methods, systems, apparatuses, and compositions.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

The “scope” of the present disclosure is defined by the claims, along with the full scope of equivalents to which such claims are entitled. The scope of the disclosure is further qualified as including any possible modification to any of the aspects and/or embodiments disclosed herein which would result in other embodiments, combinations, sub-combinations, or the like that would be obvious to those skilled in the art.

### Discussion of the Technology

Disclosed herein are new compositions and methodologies for synergistically improving needle-based local delivery of polymer-assisted ethanol ablation by introducing a photosensitizing agent, which enables visualization of the local drug distribution volume. Ethanol ablation involves the injection of ethanol into tissues to cause cell cytoplasmic dehydration and subsequent necrosis and is a cost-effective technique to treat a variety of diseases, such as hepatocellular carcinomas and thyroid carcinomas. Polymer-assisted delivery of ethanol with ethyl cellulose (EC) is utilized herein to limit ethanol leakage and further increase local necrosis. As described herein, EC-ethanol is combined with a photosensitizer to enable the visualization, through fluorescence imaging, of EC-ethanol distribution in tissues. Beneficially, the photosensitizer also enables photodynamic therapy applications. Photodynamic therapy involves delivering photosensitizers to a region of interest and activating them with a light source, such as near-infrared light, to generate reactive oxygen species (e.g., singlet oxygen) that can induce cell death. The activated photosensitizers also emit fluorescence signals for imaging the depot within the tissue platform.

### Compositions

The formulations disclosed herein beneficially combine a solvent, preferably an alcohol such as ethanol; a polymer, such as ethyl cellulose; a photosensitizer such as a benzoporphyrin derivative (BPD); and optionally one or more drugs (therapeutic agents) to enable visualization of drug delivery and/or disease treatment via photodynamic therapy. Red light activation of the compositions, for example, BPD in EC-Ethanol (BPD-EC-Ethanol, BPD-EC-EtOH, LASEIT), not only generates fluorescent signals for monitoring of local EC-Ethanol distribution in tissues but also produces singlet oxygen for potential therapeutic benefits.

### Solvent

The compositions disclosed herein include at least one solvent. Preferably, the at least one solvent has ablation capabilities and where more than one solvent is present at least one of the solvents preferably has ablation capabilities.

Suitable solvents include, but are not limited to phenol, methanol, ethanol, dehydrated alcohol (ABLYSINOL®), absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, urea, lipiodol, a combination of ethanol-glycerol, polidocanol, or a combination thereof.

In a preferred embodiment, the solvent comprises an alcohol. As used herein, the term “alcohol” refers to any compound characterized by one or more hydroxyl (—OH) groups attached to a carbon atom of an alkyl group, according to the general formula R—OH, wherein R is an alkyl group. Preferably, the alkyl group is a C₁-C₂₀ branched or unbranched alkyl group. In a further preferred embodiment, the alcohol is one according to the formula R—OH, wherein R is a C₂ branched or unbranched alkyl group. More preferably, the alcohol is ethyl alcohol.

In an embodiment, the solvent may comprise from about 0.1 wt. % to about 99 wt. % of the composition, for example, 0.5 wt. %, 1 wt. %, 3 wt. %, 4 wt. %, 5 wt. %, 6 wt. %, 7 wt. %, 8 wt. %, 9 wt. %, 10 wt. %, 11 wt. %, 12 wt. %, 13 4.%, 14 wt. %, 15 wt. %, 16 wt. %, 17 wt. %, 18 wt. %, 19 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 33 wt. %, 34 wt. %, 35 wt. %, 36 wt. %, 37 wt. %, 38 wt. %, 39 wt. %, 40 wt. %, 41 wt. %, 42 wt. %, 43 wt. %, 44 wt. %, 45 wt. %, 46 wt. %, 47 wt. %, 48 wt. %, 49 wt. %, 50 wt. %, 51 wt. %, 52 wt. %, 53 wt. %, 54 wt. %, 55 wt. %, 56 wt. %, 57 wt. %, 58 wt. %, 59 wt. %, 60 wt. %, 61 wt. %, 62 wt. %, 63 wt. %, 64 wt. %, 65 wt. %, 66 wt. %, 67 wt. %, 68 wt. %, 69 wt. %, 70 wt. %, 71 wt. %, 72 wt. %, 73 wt. %, 74 wt. %, 75 wt. %, 76 wt. %, 77 wt. %, 78 wt. %, 79 wt. %, 80 wt. %, 81 wt. %, 82 wt. %, 83 wt. %, 84 wt. %, 85 wt. %, 86 wt. %, 87 wt. %, 88 wt. %, 89 wt. %, 90 wt. %, 91 wt. %, 92 wt. %, 93 wt. %, 94 wt. %, 95 wt. %, 96 wt. %, 97 wt. %, 98 wt. %, or 99 wt. %. inclusive of all integers within these ranges.

### Polymer

The compositions disclosed herein include at least one polymer. Preferably, the at least one polymer interacts with the solvent to suspend the other components of the composition (e.g., the photosensitizer or therapeutic agent) such that upon injection to a target tissue or cell(s), the composition exhibits minimal crack formation. Additionally, the polymer preferably has a high refractive index to propagate light and further preferably aids in photosensitizer distribution. In an embodiment, at least one polymer interacts with the solvent to increase the viscosity of the composition, preferably such that the composition takes the form of a gel.

In an embodiment, the polymer has an average molecular weight of 2,000 to 100,000 daltons. In a preferred embodiment, the polymer is a cellulosic polymer. The term “cellulosic” refers to a cellulosic polymer (cellulose-containing, cellulose-derived) that has been modified by a reaction of at least a portion of the hydroxyl groups on the saccharide repeat units with a compound to form ester-linked or ether-linked substituents. For example, cellulosic ethyl cellulose has ether-linked ethyl substituents attached to the saccharide repeat units, while cellulosic cellulose acetate has ester-linked acetate substituents. Suitable examples include, but are not limited to cellulose, methyl cellulose, ethyl cellulose, ethyl methyl cellulose, hydroxy ethyl cellulose, methyl cellulose, sodium carboxy methyl cellulose, benzyl cellulose, carboxy methyl cellulose, cellulose acetate, hydroxy propyl cellulose, hydroxypropyl methyl cellulose, cellulose gum, crosslinked sodium carboxy methyl cellulose, enzymatically hydrolyzed carboxy methyl cellulose, or a combination thereof.

Particularly preferred polymers include ethyl cellulose (EC), methyl ethyl cellulose (MEC), carboxymethyl cellulose (CMC), carboxymethyl ethyl cellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), ethyl hydroxyethyl cellulose (EHEC), cyanoethyl cellulose, alginate, chitosan, or a combination thereof.

The polymer is preferably provided in solution. When provided in solution, the polymer is preferably present in a concentration of between about 1% (w/v) to about 15% (w/v) polymer-ethanol, for example, 1%, 2%, 3%, 4%, 5%, 6%, 7%, 8%, 9%, 10%, 11%, 12%, 13%, 14%, or 15%, (w/v), inclusive of all integers within this range.

### Photosensitizer

The compositions preferably comprise one or more photosensitizers, which are compounds that absorb light energy. The photosensitizer preferably can absorb light from about 400 nm to about 1000 nm, inclusive of all integers within this range (e.g., 400 nm, 401 nm, 402 nm, etc.). In an embodiment, the photosensitizer has low solubility and high permeability, or low solubility and low permeability.

The photosensitizer can be any composition that absorbs light and initiates a photochemical reaction that produces cytotoxic products. For example, suitable photosensitizers that can be used include, but are not limited to, hematoporphyrins, photofrins, chlorins such as meta-tetra hydroxyphenyl chlorin, mono-L-aspartyl chlorin e6, or bacteriochlorins, or derivatives thereof. The photosensitizer can also include phthalocyanines, porphyrins, benzoporphyrins, 5-aminolevulinic acid (ALA), or derivatives thereof. Other photosensitizers include, but are not limited to, purpurins, porphycenes, pheophorbides, and verdins. Purpurins are a class of porphyrin macrocycle with an absorption band at from about 630 nm to about 715 nm, typified by tin etiopurpurin (SnET2), which has an extinction coefficient of 40,000 M⁻¹ cm⁻¹ at about 700 nm. Porphycenes, having activation wavelengths of about 635 nm, are also useful. Phorbides are derived from chlorophylls (e.g. pheophorbide) and are also useful as photosensitizers. Verdins contain a cyclohexanone ring fused to one of the pyrroles of the porphyrin ring and can also be used as a photosensitizer. Psoralens are another example of a photosensitizer that can be used in the disclosed conjugates and methods.

In a preferred embodiment, the photosensitizer comprises a porphyrin. Suitable porphyrin photosensitizers include but are not limited to, hematoporphyrin and derivatives thereof (HpD), photofrin, verteporfin, or a combination thereof. Further discussion of porphyrin photosensitizers is found in Kou et al., Porphyrin photosensitizers in photodynamic therapy and its applications, ONCOTARGET, 2017; 8(46): 81591-81603, which is herein incorporated by reference in its entirety. In a preferred embodiment, the photosensitizer is free-form verteporfin (VP) powder.

In an embodiment, the photosensitizer comprises a second-generation photosensitizer designed to meet a specific demand, such as a benzoporphyrin derivative monoacid ring A (BPD-MA) verteporfin, meso-tetrakis (4-sulfonatophenyl) porphyrin (TPPS), N-aspartyl chlorin e6 NPe6, aminolevulinic acid (5-ALA), ALA (Levulan), Methyl-ALA (Metvix), hexyl-ALA, temoporfin or m-THPC, TSPP, HPPH (Photoclor), hypericin, or a combination thereof. Alternatively, the photosensitizer may comprise a third-generation photosensitizer such as chlorin E6 (Ce6), chlorin e6 trisodium, Npe6, mono-L-aspartyl chlorine e6, Talaporfin sodium (LST11). Redaporfin, mTHPC (Temoporfin, Foscan), Photofrin, Phthalocyanines (Photosens, or a combination thereof.

Synthetic non-porphyrin compounds can also be used as photosensitizers in the compositions and methods disclosed herein. Suitable non-porphyrin compounds include, but are not limited to, phenothiazinium compounds such as methylene blue, Toluidine blue, a cyanine such as Merocyanine-540, an acridine dye, Nile blue, and/or rhodamine such as the mitochondria-specific Rhodanine 123. The photosensitizer may also comprise a benzoporphyrin derivative, such as a benzoporphyrin mono acid derivative.

The photosensitizer may be provided in any suitable concentration, for example between 0.1 M to about 500 μM, for example 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, 110 μM, 120 ELM, 130 μM, 140 μM, 150 μM, 160 ELM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 M, or 500 μM, inclusive of all integers within these ranges. In a preferred embodiment, the concentration of photosensitizer is between about 5 μM to about 100 μM.

### Additional Optional Ingredients

The compositions described herein may optionally comprise one or more additional ingredients, such as excipients, lipids, pH modifiers, pH buffers, surfactants, or a combination thereof, to facilitate clinical use and administration.

### Excipients

The compositions optionally include one or more excipients. As used herein, the term “excipient” refers to a medium or vehicle for a drug or therapeutic agent. Excipients may include, for example, additional solvents, preservatives, fillers, or coloring agents. An excipient may be used to facilitate dissolution, prevent phase separation, maintain or modify viscosity, or preserve or stabilize a composition. Suitable examples include, but are not limited to acetone, ethanol, ethylene glycol, propylene glycol, butane-1,3-diol, isopropyl myristate, isopropyl palmitate, mineral oil, or a combination thereof

### Surfactant

The compositions optionally include one or more surfactants. Suitable surfactants include, but are not limited to polyethylene glycol (PEG) laurate, Tween 20, Tween 40, Tween 60, Tween 80, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, plyglyceryl-6 oleate, polyglyceryl-6 myristate, polyglyceryl-6 palmitate, polyglyceryl-10, laurate, plyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG lauryl ether, organic acid, salts of any organic acid and organic amine, polyglycidol, glycerol, glycerol, multiglycerols, galactitol, di(ethylene glycol), tri(ethylene glycol), tetra(ethylene glycol), penta(ethylene glycol), poly(ethylene glycol) oligomers, di(propylene glycol), tri(propylene glycol), tetra(propylene glycol), penta(propylene glycol), poly(propylene glycol) oligomers, a block copolymer of polyethylene glycol and polypropylene glycol, Pluronic, Pluronic 85, and derivatives and combinations thereof. In some embodiments, the content of the surfactant in the formulation may range from 0.1% by weight to 80% by weight, from 0.5% by weight to 50% by weight, or from 1% by weight to 15% by weight.

### Lipids

In one embodiment, the compositions optionally include one or more of an oil, a fatty acid, and/or a lipid. The at least one of an oil, a fatty acid, and a lipid in the formulation is chosen from butanoic acid, hexanoic acid, octanoic acid, decanoic acid, dodecanoic acid, tetradecanoic acid, hexadecanoic acid, octadecanoic acid, octadecatrienoic acid, eicosanoic acid, eicosenoic acid, eicosatetraenoic acid, eicosapentaenoic acid, docosahexaenoic acid, tocotrienol, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, natural or synthetic phospholipids, mono-, di-, or triacylglycerols, cardiolipin, phosphatidylglycerol, phosphatidic acid, phosphatidylcholine, alpha tocoferol, phosphatidylethanolamine, sphingomyelin, phosphatidylserine, phosphatidylinositol, dimyristoylphosphatidvlcholine, dioleoylphosphatidylcholine, dipalmitoylphosphatidylcholine, distearoylphosphatidylcholine, phosphatidylethanolamines, phosphatidylglycerols, sphingolipids, prostaglandins, gangliosides, neobee, niosomes, and derivatives thereof

### Additional Drug or Therapeutic Agent

As described herein, the photosensitizer and/or solvent (e.g., ethanol) provide therapeutic efficacy, particularly antitumor efficacy. Optionally, the compositions may comprise one or more additional drugs or therapeutic agents in addition to the photosensitizer and solvent. For example, the therapeutic agent may comprise an immunotherapeutic agent. Examples of suitable immunotherapeutic agents include, without limitation, a checkpoint inhibitor, monoclonal antibody, cytokine, or a combination thereof. The therapeutic agent may comprise a gene therapy agent. A gene therapy agent includes any nucleic acid construct capable of transforming or impacting one or more cells. Examples of suitable gene therapy agents include, without limitation, a plasmid, viral vector, RNAi molecule, RNA construct, DNA construct, gene product (i.e., a protein encoded by a nucleic acid construct that, when expressed in the cell, has an effect on the cell), promoter, or a combination thereof.

The additional drug may comprise an antineoplastic drug comprising, for example, emaxanib, cyclosporin, etanercept, doxycycline, bortezomib, acivicin, aclarubicin, acodazole hydrochloride, acronine, adozelesin, aldesleukin, altretamine, ambomycin, ametantrone acetate, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, celecoxib, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, iproplatin, irinotecan, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, taxotere, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

The additional therapeutic agent may comprise, for example, 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chlorins, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, 9-, dioxamycin, diphenyl spiromustine, docetaxel, docosanol, dolasetron, doxifluridine, doxorubicin, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imatinib (Gleevec®), imiquimod, immunostimulant peptides, insulin-like growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mitoguazone, mitolactol, mitomycin analogues, mitonafide, mitotoxin fibroblast growth factor-saporin, mitoxantrone, mofarotene, molgramostim, Erbitux, human chorionic gonadotrophin, monophosphoryl lipid A+mycobacterium cell wall sk, mopidamol, mustard anticancer agent, mycaperoxide B, mycobacterial cell wall extract, myriaporone, N-acetyldinaline, N-substituted benzamides, nafarelin, nagrestip, naloxone/pentazocine, napavin, naphterpin, nartograstim, nedaplatin, nemorubicin, neridronic acid, nilutamide, nisamycin, nitric oxide modulators, nitroxide antioxidant, nitrullyn, oblimersen, 06-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras famesyl protein transferase inhibitors, ras inhibitors, ras-GAP inhibitor, retelliptine demethylated, rhenium Re 186 etidronate, rhizoxin, ribozymes, RII retinamide, rohitukine, romurtide, roquinimex, rubiginone Bl, ruboxyl, safingol, saintopin, SarCNU, sarcophytol A, sargramostim, Sdi 1 mimetics, semustine, senescence derived inhibitor 1, sense oligonucleotides, signal transduction inhibitors, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stipiamide, stromelysin inhibitors, sulfinosine, superactive vasoactive intestinal peptide antagonist, suradista, suramin, swainsonine, tallimustine, tamoxifen methiodide, tauromustine, tazarotene, tecogalan sodium, tegafur, tellurapyrylium, telomerase inhibitors, temoporfin, teniposide, tetrachlorodecaoxide, tetrazomine, thaliblastine, thiocoraline, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, velaresol, veramine, verdins, vinorelbine, vinxaltine, vitaxin, vorozole, zanoterone, zeniplatin, zilascorb, zinostatin stimalamer, or a combination thereof.

### Methods of Making

The present disclosure also relates to methods of making the therapeutic compositions described herein. In an embodiment, the method comprises dissolving a polymer in a solvent to form a polymer-solvent solution; dissolving a photosensitizer in the polymer-solvent solution; and optionally adding one or more drugs or therapeutic agents to the composition. In some embodiments, the final solution comprises between about 0.10% w/v to 99% w/v of solvent, inclusive of all integers within this range.

In a preferred embodiment, the photosensitizer is a benzoporphyrin derivative (BPD). In such an embodiment, to prepare the BPD stock solution, 5 mg of the BPD powder is dissolved with 1 mL of dimethyl sulfoxide (DMSO). The molarity of the sample may be confirmed with a microplate reader using the established molar extinction coefficient of BPD in dimethyl sulfoxide (DMSO; ˜34,895 M⁻¹ cm⁻¹ at 687 nm).

According to an embodiment, preparation of a SOSG stock solution comprises dissolving 100 μg of the SOSG powder with 33 μL of methanol in the original container, resulting in a 5 mM SOSG concentration. The container was wrapped in aluminum foil and stored in a −20° C. freezer. The reconstituted solution was generally stable at −20° C. for several months but should only be at room temperature (25° C.) for a maximum of 4 hours.

In a further preferred embodiment, the photosensitizer is a benzoporphyrin derivative (BPD), the polymer is ethyl cellulose, and the solvent is ethanol. In an embodiment, to prepare the BPD-EC-Ethanol solution, 0.9468 g EC was dissolved in 20 mL pure ethanol to form a 6% EC-Ethanol (w/v) stock solution. Next, 1500 μL of the 6% EC-Ethanol solution was transferred to a 2 mL microcentrifuge tube, and enough stock BPD solution was added to create the BPD-EC-Ethanol solution. For preparing solutions with different BPD concentrations, the volume of stock BPD solution can be changed as required. For example, with an 8 mM stock BPD solution, 3.75 μL of the stock BPD solution was added to 1494.75 μL of the 6% EC-Ethanol solution to create a 20 μM BPD-EC-Ethanol solution (BPD to EC-Ethanol, v/v). Then, 1.5 μL of SOSG stock solution was added to the BPD-EC-Ethanol solution, resulting in a 5 μM SOSG solution in BPD-EC-Ethanol. The tube was inverted to mix the contents until the solution was homogenized. The BPD gave the solution a light-green color.

### Methods of Administration

The method of administration will vary depending on the particular use, e.g., whether as part of cancer therapy, drug delivery, imaging, etc. In a preferred embodiment, however, the compositions are administered via injection, preferably percutaneous injection. Accordingly, although the compositions may be provided in any form (e.g., tablet, capsule, aerosol, etc.) they are preferably provided in the form of a liquid.

Administration of the compositions may occur manually or with the assistance of an electrical device. Beneficially, the compositions described herein may be administered in a flow rate, depth, and volume that permit manual administration. Thus, in a preferred embodiment, administration occurs manually with a needle and syringe. For example, minimally invasive ablation devices comprising an injection needle and control by an electrical device (e.g., injector) may be introduced to the target site using a trocar inserted through a small opening formed in the subject's body or through a natural body orifice such as the mouth, anus, or vagina using translumenal access. Once the ablation devices are located in or proximal to the undesirable tissue in the treatment region, the electrical device may be activated to administer the compositions. The ablation devices comprise portions that may be inserted into the tissue treatment region percutaneously (e.g., where access to inner organs or other tissue is done via needle-puncture of the skin). Other portions of the ablation devices may be introduced into the tissue treatment region endoscopically (e.g., laparoscopically and/or thoracoscopically) through trocars or channels of the endoscope, through small incisions, or transcutaneously.

The light source used to activate the photosensitizer in the compositions can be provided by any suitable light source. For example, the light can be in the form of a laser or in the form of a fiber optic source used to deliver light to the treatment site from a laser. Beneficially, the compositions described herein propagate the light source, enabling the light source to reach a greater depth in the target than would be possible without the compositions of the disclosure. In an embodiment, the light source reaches a depth in the target of between about 0 cm to about 1.5 cm in the target, i.e., up to about 1.5 cm, including for example 0.1 cm, 0.2 cm, 0.3 cm, 0.4 cm, 0.5 cm, 0.6 cm, 0.7 cm, 0.8 cm, 0.9 cm, 1.0 cm, 1.1 cm, 1.2 cm, 1.3 cm, 1.4 cm, or 1.5 cm, inclusive of all integers within this range.

The administration may occur at any suitable injection rate. In an embodiment, the administration occurs at an injection rate of between about 10 mL/hour to about 100 mL/hour, including for example, 10 mL/hour, 11 mL/hour, 12 mL/hour, 13 mL/hour, 14 mL/hour, 15 mL/hour, 16 mL/hour, 17 mL/hour, 18 mL/hour, 19 mL/hour, 20 mL/hour, 21 mL/hour, 22 mL/hour, 23 mL/hour, 24 mL/hour, 25 mL/hour, 26 mL/hour, 27 mL/hour, 28 mL/hour, 29 mL/hour, 30 mL/hour, 31 mL/hour, 32 mL/hour, 33 mL/hour, 34 mL/hour, 35 mL/hour, 36 mL/hour, 37 mL/hour, 38 mL/hour, 39 mL/hour, 40 mL/hour, 41 mL/hour, 42 mL/hour, 43 mL/hour, 44 mL/hour, 45 mL/hour, 46 mL/hour, 47 mL/hour, 48 mL/hour, 49 mL/hour, 50 mL/hour, 51 mL/hour, 52 mL/hour, 53 mL/hour, 54 mL/hour, 55 mL/hour, 56 mL/hour, 57 mL/hour, 58 mL/hour, 59 mL/hour, 60 mL/hour, 61 mL/hour, 62 mL/hour, 63 mL/hour, 64 mL/hour, 65 mL/hour, 66 mL/hour, 67 mL/hour, 68 mL/hour, 69 mL/hour, 70 mL/hour, 71 mL/hour, 72 mL/hour, 73 mL/hour, 74 mL/hour, 75 mL/hour, 76 mL/hour, 77 mL/hour, 78 mL/hour, 79 mL/hour, 80 mL/hour, 81 mL/hour, 82 mL/hour, 83 mL/hour, 84 mL/hour, 85 mL/hour, 86 mL/hour, 87 mL/hour, 88 mL/hour, 89 mL/hour, 90 mL/hour, 91 mL/hour, 92 mL/hour, 93 mL/hour, 94 mL/hour, 95 mL/hour, 96 mL/hour, 97 mL/hour, 98 mL/hour, 99 mL/hour, or 100 mL/hour.

The administration may occur at any suitable injection volume, based on the size and type of the target. In an embodiment, the administration occurs at an injection volume of between about 100 μL to about 5000 μL, including for example, 100 μL, 105 μL, 110 μL, 115 μL, 120 μL, 125 μL, 130 μL, 135 μL, 140 μL, 145 μL, 150 μL, 155 μL, 160 μL, 165 μL, 170 μL, 175 μL, 180 μL, 185 μL, 190 μL, 195 μL, 200 μL, 205 μL, 210 μL, 215 μL, 220 μL, 225 μL, 230 μL, 235 μL, 240 μL, 245 μL, 250 μL, 255 μL, 260 μL, 265 μL, 270 μL, 275 μL, 280 μL, 285 μL, 290 μL, 295 μL, 300 μL, 305 μL, 310 μL, 315 μL, 320 μL, 325 μL, 330 μL, 335 μL, 340 μL, 345 μL, 350 μL, 355 μL, 360 μL, 365 μL, 370 μL, 375 μL, 380 μL, 385 μL, 390 μL, 395 μL, 400 μL, 405 μL, 410 μL, 415 μL, 420 μL, 425 μL, 430 μL, 435 μL, 440 μL, 445 μL, 450 μL, 455 μL, 460 μL, 465 μL, 470 μL, 475 μL, 480 μL, 485 μL, 490 μL, 495 μL, 500 μL, 600 μL, 700 μL, 800 μL, 900 μL, 1000 μL, 1100 μL, 1200 μL, 1300 μL, 1400 μL, 1500 μL, 1600 μL, 1700 μL, 1800 μL, 1900 μL, 2000 μL, 2100 μL, 2200 μL, 2300 μL, 2400 μL, 2500 μL, 2600 μL, 2700 μL, 2800 μL, 2900 μL, 3000 μL, 3100 μL, 3200 μL, 3300 μL, 3400 μL, 3500 μL, 3600 μL, 3700 μL, 3800 μL, 3900 μL, 4000 μL, 4100 μL, 4200 μL, 4300 μL, 4400 μL, 4500 μL, 4600 μL, 4700 μL, 4800 μL, 4900 μL, or 5000 μL, inclusive of all integers within this range.

The administration may occur at any suitable insertion depth, based on the size, type, and depth of the target. In an embodiment, the administration occurs at an insertion depth of between about 4 mm to about 120 mm, including for example, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm, 100 mm, 101 mm, 102 mm, 103 mm, 104 mm, 105 mm, 106 mm, 107 mm, 108 mm, 109 mm, 110 mm, 111 mm, 112 mm, 113 mm, 114 mm, 115 mm, 116 mm, 117 mm, 118 mm, 119 mm, or 120 mm, inclusive of all integers within this range.

Administration may occur at any suitable interval based on the target or disease. For example, the administration may be scheduled to coincide with the administration of other therapeutic agents or drugs so as to simplify drug delivery. In an embodiment, administration frequency (i.e., how often administration occurs in a given period) occurs between once a week and once a month, including for example once each week, once every other week/twice a month, or once a month. The administration period (i.e., a cycle of treatment) may last over a period of between about three months to about one year, including for example three months, four months, five months, six months, seven months, eight months, nine months, ten months, eleven months, or twelve months/one year. The administration period may be repeated as needed (for example the period may be repeated between one and ten times), optionally with gaps of time in between each period (for example, a time gap of between one week and three months in between each administration period).

### Methods of Use in Ablation & Photodynamic Therapy

In an embodiment, the photosensitizer(s) in the compositions function as an antineoplastic drug, and specifically as a chemotherapeutic agent when administered as part of photodynamic therapy. Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Current cancer therapy may involve surgery, chemotherapy, hormonal therapy, radiation treatment, biological therapy, and/or immunotherapy to eradicate neoplastic cells. The nanoparticles and compositions described herein may be used as part of cancer therapy, wherein the cancer includes, without limitation, peritoneal cancer, liver cancer, pancreatic cancer, brain cancer (e.g., glioblastoma treatment), neck cancer, spinal cancer, lung cancer, prostate cancer, bladder cancer, skin cancer, eye cancer, oral cancer, head and neck cancer, breast cancer, blood cancer, bone cancer, stomach cancer, kidney cancer, colorectal cancer, cervical cancer, ovarian cancer, central nervous system tumor, or a combination thereof.

Provided herein are methods of treating a disease, particularly cancer through PDT, comprising (a) administering to a target location the compositions described herein comprising a photosensitizer in an amount effective to facilitate photodynamic therapy (PDT) and (b) exposing the compositions photoactivating light having a wavelength capable of being absorbed by the photosensitizer; thereby (c) producing cytotoxic reactive oxygen species at the target location. In an embodiment, the target location is a tumor, a tissue, a cell, a vessel, or a combination thereof.

In an embodiment the light source or energy source comprises electromagnetic, kinetic, thermal, mechanical, oscillation, ultrasound, magnetic resonance, radio frequency, magnetic, laser, vibrational, thermal, cryotherapy, high intensity focused ultrasound, acoustic, optical-acoustic ultrasound, microwave, radioactive, x-ray, irreversible electroporation, electric current, electro cautery, chemical, electro chemical, mechanical chemical, ultraviolet light, infrared light, and/or visible light. Examples of irradiation devices and methods of using the same can be found in U.S. Pat. No. 9,974,974, which is herein incorporated by reference in its entirety.

Also provided herein are methods of treating a disease, particularly cancer, through the use of the photosensitizer as a chemotherapeutic agent, the method comprising administering to a target an effective amount of a composition comprising a photosensitizer. In an embodiment, the target is located in a mammal, particularly a human. In an embodiment, the target comprises a tumor, a tissue, a cell, a vessel, or a combination thereof.

In an embodiment, the composition further comprises or is administered in conjunction with an additional drug or therapeutic agent besides the photosensitizer and solvent (e.g., ethanol).

In an embodiment, the compositions disclosed herein are also used to kill target tissue or cells through ablation. Ablation, namely ethanol ablation, involves the injection of ethanol directly into a target site to kill target cells. Ethanol ablation, also referred to as percutaneous ethanol injection (PEI), is relatively simple to perform, the method generally comprising injecting ethanol (e.g., absolute alcohol) through a needle placed percutaneously directly into a target site, such as a tumor. The necrosis produced by ethanol injection results from cellular dehydration and tissue ischemia from vascular thrombosis. Ethanol ablation may also be considered for recurrent or partially treated disease previously managed with an alternative minimally invasive technique.

Ethanol ablation may be used to treat cancer or a precancerous lesion, a cancerous lesion, a or a benign lesion or growth (e.g., a benign epithelial lesion such as a benign lesion on the skin such as a wart or skin tag) in a subject, comprising administering to the subject a therapeutically effective amount of the compositions described herein, such that the cancer or lesion is reduced in size or the number of living cells in the cancer or lesion are completely or partially reduced, compared to the cancer or lesion prior to treatment. Ablation may also be used to ameliorate a lesion or growth in a subject, the method comprising, consisting of, or consisting essentially of administering to the subject a therapeutically effective amount of a therapy solution such that the lesion is ameliorated.

In a preferred embodiment, the compositions are used in combination ablation therapy and photodynamic therapy to provide synergistic efficacy compared to either method of therapy alone.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of embodiments, and without departing from the spirit and scope thereof, can make various changes and modifications of the embodiments to adapt to various usages and conditions. Thus, various modifications of the embodiments, in addition to those shown and described herein, will be apparent to those skilled in the art from the foregoing description. Such modifications are also intended to fall within the scope of the appended claims.

### Materials Used

The following is non-exclusive listing of materials and equipment used in the examples:
20-dram polystyrene container (with snap cap)
30-dram polystyrene container (with snap cap)
0.5-10 μL single channel pipette
100-1000 μL single channel pipette
2 mL roundbottom, self-standing cryovial
2 mL microcentrifuge tube
Mass balance, 220 g
Weigh boat
Stirring hotplate
Stir bar
Surgical scissors
Razor blade
Laboratory Spatula
Deionized water
Thin Wall Heat Shrink Tubing
96-well, clear flat bottom microplate
Kimwipes
Paper towel
1000 mL glass bottle (with cap)
−20° C. freezer
Verteporfin
Singlet Oxygen Sensor Green
Ethyl cellulose
Ethanol, 200 proof
Dimethyl sulfoxide
Methanol
Ultrapure Agarose
Swine liver block
Nanomite Syringe pump
1 mL syringe
3 mL syringe
27-gauge needle
Support Rod and Base
Talon Clamp
Clamp Holder
Laboratory jack
Microplate Reader
Continuous Wave Laser
685 nm TO-can laser diode
50 mm collimating lens
Objective lens
SMA-905 connector
735/728 nm bandpass filter
12-bit CCD camera
Microsoft Office
MATLAB
ImageJ Fiji

### Example 1. Formulation and Evaluation of the Light-Activatable, Sustained-Exposure Compositions

### Phantom Tissue Preparation

In this protocol, phantom tissues were prepared for the BPD-EC-ethanol injection, and the backflow quantification and distribution visualization were performed. For the tumor-mimicking agarose mechanical phantom, 10 g of ultrapure agarose was dissolved in 1000 mL of deionized water on a hot stir plate, heated at 200° C. while stirring for 3 hours, and then distributed into polystyrene containers at approximately 80% total volume and stored at 4° C. for 24 hours. For the swine liver tissue, a 30-dram polystyrene container was used, and a 3″×4″×2″ section was cut out with surgical scissors and placed into the container. The liver section was immobilized by filling the remaining space in the container.

### BPD-EC-Ethanol Injection Setup and Protocol

Next, for the BPD-EC-Ethanol injection, a 3 mL syringe containing the BPD-EC-ethanol solution was loaded onto a programmable syringe pump, and a syringe needle (27G beveled) was attached to the end of the syringe. The desired injection parameters were entered into the syringe pump, and the needle was inserted straight into the phantom (minimizing crack formation) and also into the swine liver section. The injection was started, and the needle was left in place for 5 minutes before removal to ensure that all the programmed volumes were infused into the phantom or tissue. Further details regarding general injection protocol, is found in Morhard et al., Understandingfactors governing distribution volume of ethyl cellulose-ethanol to optimize ablative therapy in the liver, IEEE transactions on bio-medical engineering. 67 (8), 2337-2348 (2020), which is herein incorporated by reference in its entirety.

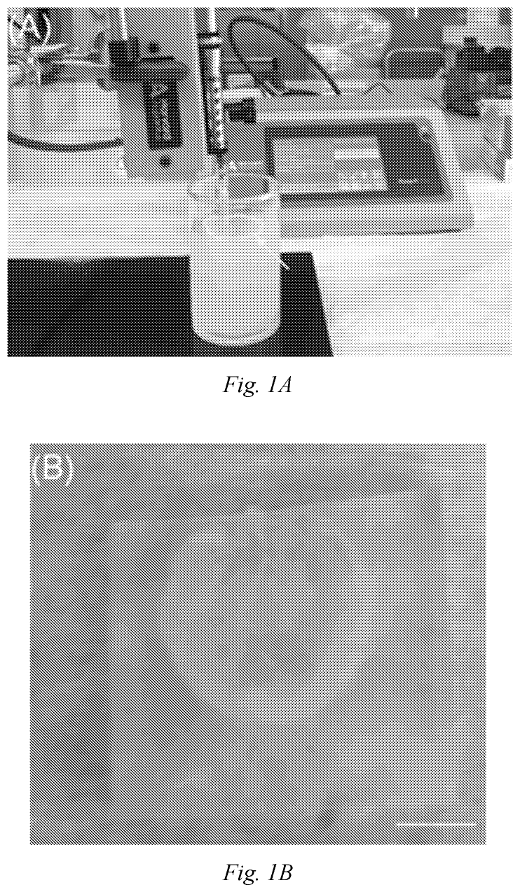

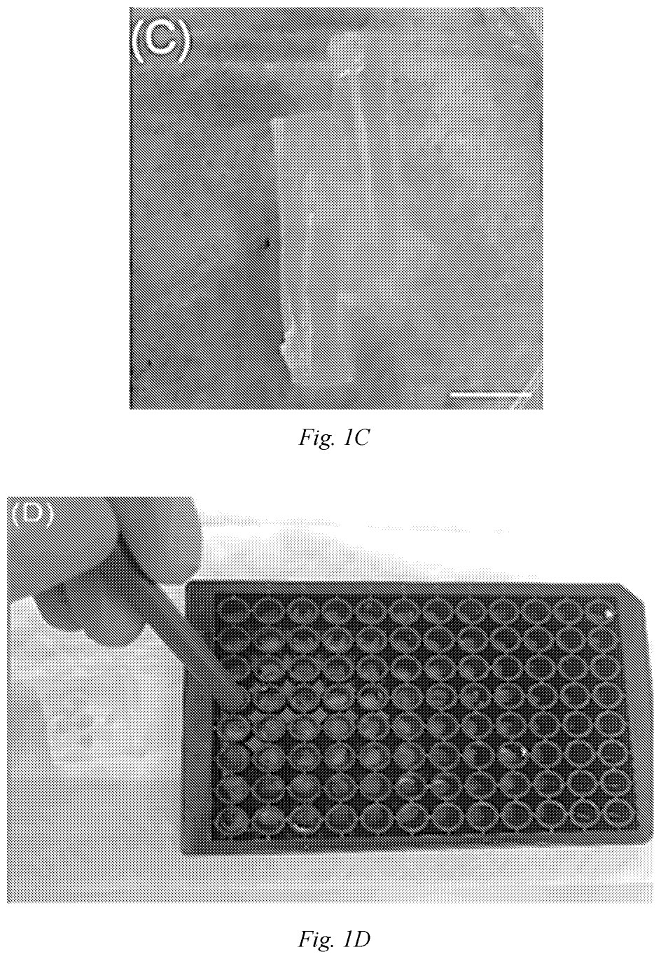

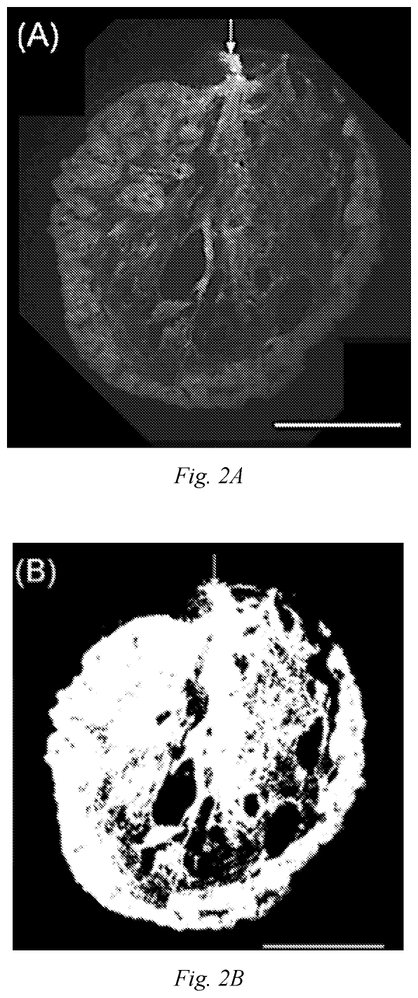

The injection setup and method of execution to obtain a distribution volume for analysis are shown in FIGS. 1A-1D. The prepared BPD-EC-Ethanol formulation was primed by the syringe pump and injected into the sample, with gelated retrograde backflow (yellow arrow, outline in the sample container) collected shortly afterward for volume measurements (FIG. 1A). After removing the sample from its container, any excess sample was removed to expose the frontal plane of the distribution (FIG. 1B) for fluorescence imaging. To measure the distribution volume, the sample distribution was then bisected along the medial plane to expose the side cross-sectional areas for subsequent fluorescence imaging (FIG. 1C). To characterize the photochemical and photophysical properties of BPD, punch biopsies spanning the entirety of the sample (e.g., sourced from FIG. 1B) were collected and placed into a microplate for irradiance and fluorescence analysis (FIG. 1D).

### BPD-EC-Ethanol Backflow Quantification

For backflow quantification, the mass of empty microcentrifuge tubes designated for each injection sample was measured. After completion of the injection and removal of the needle, any residual BPD-EC-ethanol that gelated on the tissue surface was removed and placed into their respective microcentrifuge tubes. The mass of the tubes containing the residual BPD-EC-ethanol was measured, and the difference between this mass and that of the empty tubes was the total backflow mass from the injection. The total backflow mass was then mathematically converted to volume using the density of ethanol (0.789 g/mL). The composition of the BPD-EC-Ethanol formulation is predominantly pure ethanol solution, and thus beneficially, the contributions of BPD and EC to the overall density of the BPD-EC-Ethanol are understood to be negligible.

Table 1 details a strategy for measuring and calculating retrograde backflow leakage from an injection. One replicate was provided to demonstrate how to calculate the volume of backflow from a single injection of 300 μL of BPD-EC-Ethanol into both a phantom and tissue. A pre-massed microcentrifuge tube was used to determine the mass of the backflow, which was then converted to volume, given the density of ethanol (0.789 g/mL). This strategy can be applied to quantify retrograde backflow leakage observed in other liquid needle injections.

TABLE 1
Retrograde backflow leakage calculation.

| | | | | | Backflow |
|---|---|---|---|---|---|
| | Empty | Tube | | | volume/ |
| | tube | mass + | | Backflow | Infusion |
| | mass | backflow | Backflow | volume | volume |
| Sample | (g) | mass (g) | mass (g) | (mL) | ratio (%) |
| Phantom | 1.098 | 1.208 | 0.110 | 0.139 | 46.5% |
| Tissue | 1.129 | 1.137 | 0.008 | 0.010 | 3.4% |

### BP-EC-Ethanol Distribution Visualization

For BP-EC-Ethanol distribution visualization in the tumor-mimicking mechanical phantom, excess phantom was removed until reaching the front cross-section area of the distribution, leaving a buffer region of a few mm around the depot. A fluorescent image of the area was taken using a top-down angled fluorescent imaging system with an excitation wavelength of 685 nm, an emission filter of 735 nm, and irradiance of 2.5 mW/cm². The cross-section area was bisected to view the distribution. The distribution characteristics and intensities of the BPD-EC-Ethanol solution were quantified via ImageJ Fiji using procedures described below.

The system used to image the tumor mimicking phantom was a custom-made fluorescent imaging setup. A TO-can laser source with a central wavelength of 685 nm was used. The laser was then collimated and illuminated on the sample at a 112.5-degree angle to the surface. The fluorescence was collected through an objective lens normal to the surface, filtered through a 735/28 nm filter, and collected by a 12-bit CCD camera.

For BPD-EC-Ethanol distribution visualization in the swine liver tissue, the liver section was removed from the dram container while taking note of the injection site on the section. Using a razor blade, the injection site was sliced through the middle, exposing the cross-section area of the distribution. A fluorescence image of this area was taken using the same parameters as detailed previously. The cross-section area was then bisected down the middle, and the two pieces were flipped to expose the thickness of the cross-section area. Another fluorescent image of this thickness was taken using the same parameters described previously. The distribution characteristics and intensities of the BPD-EC-Ethanol solution were quantified using the procedure outlined below via MATLAB (see Appendix).

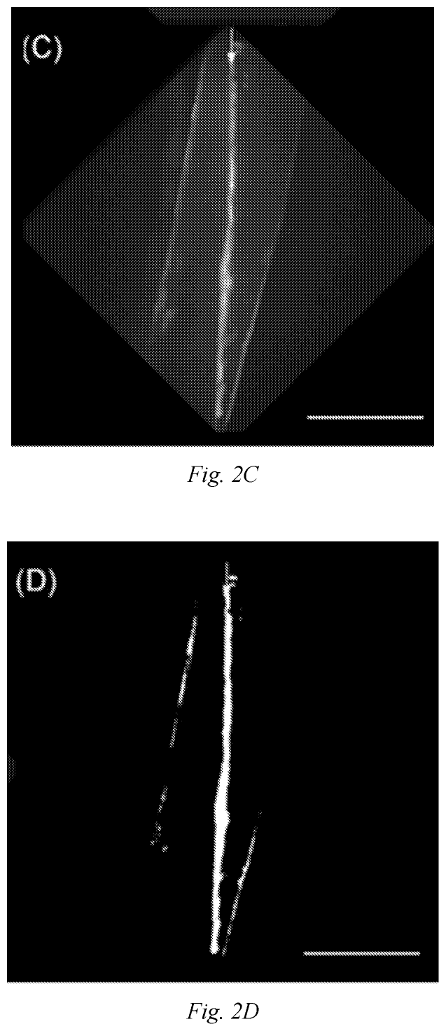

FIGS. 2A-2D show representative fluorescence images of the BPD-EC-Ethanol distribution in tumor-mimicking phantoms. Included in these figures are front and side views in the phantom (FIGS. 2A and 2C, respectively). Binary images for the phantom, obtained by automatically segmenting the images via the Otsu algorithm, are also included (FIGS. 2B and 2D, respectively). The images of the front cross-sectional areas were manually stitched together, based on the Pairwise stitching plugin. The total volume of the distribution for the phantom was calculated to be 45.8 μL. Varying infusion parameters yields a range of distribution volumes that can be compared to determine optimal parameters.

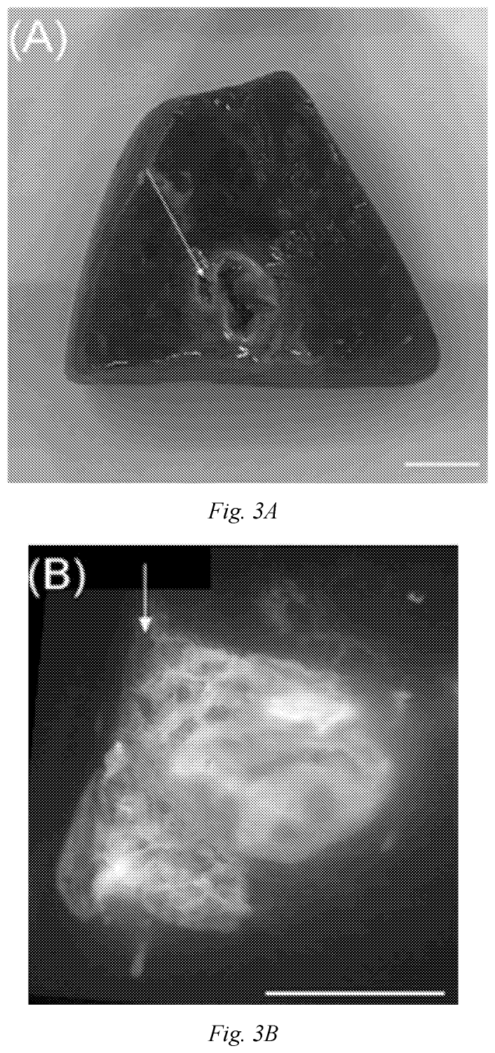

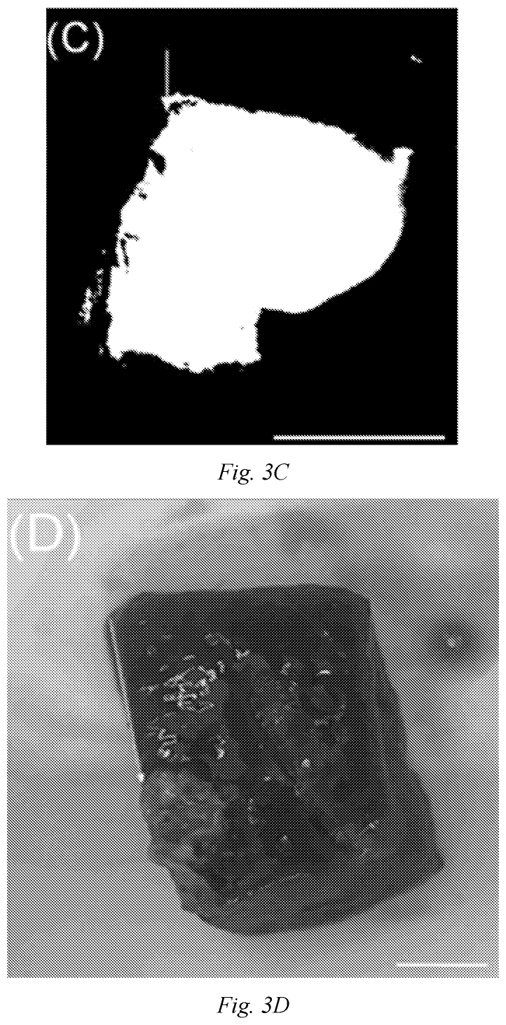

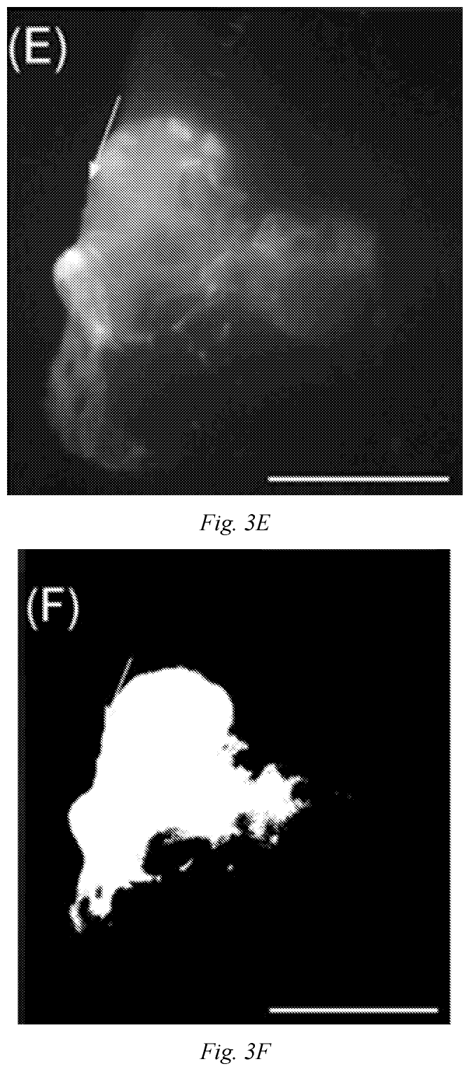

FIGS. 3A-3F show representative fluorescence images of the BPD-EC-Ethanol distribution in swine liver tissue. Included in these figures are the digital and fluorescence images of the front and side views in the tissue (FIGS. 3A and 3D for digital images of front and side views, respectively; FIGS. 3B and 3E for fluorescence images of front and side views, respectively). Binary images for the tissue, obtained by automatically segmenting the images via the Otsu algorithm, are also included (FIGS. 3C and 3F, respectively). The images of the front cross-sectional areas were manually stitched together, based on the Pairwise stitching plugin. The total volume of the distribution for the tissue was calculated to be 260.1 μL. Varying infusion parameters yields a range of distribution volumes that can be compared to determine optimal parameters.

### BPD-EC-Ethanol Distribution Volume Quantification

To quantify the distribution volume of the BPD-EC-Ethanol solution in cylindrical volumes, the fluorescence image of the cross-section area of the distribution was opened in the ImageJ Fiji application. If multiple fluorescence images of the same distribution were taken, the images were stitched together into one composite image using suitable software. A scale bar was added to the image and then converted to 8-bit. A segmentation threshold was applied to the fluorescence image to obtain binary images of the area distribution. The Otsu thresholding algorithm was used for the provided examples, searching for the threshold value that minimizes intra-class pixel intensity variance of the selected image.

For distributions with cylindrical volumes, for the cross-section area image, the polygon selection tool was used to trace around the distribution and measure the selection to obtain the physical characteristics of the distribution, ensuring that the area, mean gray value, perimeter, and area fraction radio buttons were selected before performing the measurements.

Emulating the polygon trace distribution for the original fluorescence image provided the mean gray values within the selection. The obtained value corresponded to the overall BPD intensity of the distribution. For the cross-section thickness image, the Local Thickness plugin was executed to generate a heat map containing the local thicknesses throughout the distribution. A line was drawn over the distribution containing the local thicknesses. The line was measured to calculate the mean thickness and multiplied by the area to obtain the volume of the distribution.

For distributions with ellipsoidal volumes, the MATLAB script as provided in the Appendix included herewith and titled “LiverDistributionVolume.m” was downloaded, and the .m file was opened in the current version of MATLAB. The file name and type of the cross-section area binary image and thickness binary image were provided for the “imgl” and “img2” variables, respectively. The file location of the fluorescent images to be analyzed was specified in the “folder” variable, and a name for the post-processed images was specified in the “imagename” variable. A value for the scale bar was specified in the “scalebar” variable, and the script was run.

When the first image popped up (“img1”), the view was changed so that both ends of the scale bar were clearly visible, and the Enter key was pressed. The cursor changed into crosshairs, and one end of the scale bar was single-clicked, and the other end was double-clicked. Another window popped up, containing “img1,” now calibrated to the scale bar. The distribution was traced around in one stroke. The process was repeated for the “img2” image, and the distribution volume, calculated in mm3 (equivalent to μL), was saved in the “FinalVolume” variable. The traced images were saved as the selected image type.

The results are shown in Table 2 and Table 3 below.

TABLE 2
Distribution volume of BPD-EC-Ethanol in a
phantom as a function of infusion rate.

| Injection | Injection | Distribution | Distribution volume/ |
|---|---|---|---|
| Rate | Volume | Volume | Injection |
| (mL/hr) | (μL) | (μL) | volume ratio (%) |
| 10 | 300 | 81.3 | 27.1% |
| 30 | 300 | 79.4 | 26.5% |
| 50 | 300 | 71.0 | 23.7% |

TABLE 3
Distribution volume of BPD-EC-Ethanol in
a phantom as a function of infusion volume.

| Injection | Injection | Distribution | Distribution volume/ |
|---|---|---|---|
| Rate | Volume | Volume | Injection |
| (mL/hr) | (μL) | (μL) | volume ratio (%) |
| 30 | 100 | 17.3 | 17.3% |
| 30 | 300 | 92.9 | 31.0% |
| 30 | 500 | 99.5 | 19.9% |

Table 2 and Table 3 provide a sample comparison of distribution volumes when infusion parameters (injection rate for Table 1 and injection volume for Table 2) were varied in phantoms. In the provided examples, the injection rate was varied between 10 and 50 mL/hr, while keeping injection volume constant at 300 μL; injection volume was varied between 100 μL and 500 μL while keeping the injection rate constant at 30 mL/hr. This strategy can be applied to determine which infusion parameters yield the maximal distribution volume in tissue, which can be extrapolated to later ex vivo and in vivo studies. In the provided example, the injection rates tested do not impact the distribution volume, but the injection volumes tested showed a higher depot volume retention for the 300 μL volume injection than the other two conditions. This example suggests that distribution volume is directly impacted by injection volume but is less sensitive to changes in injection rate.

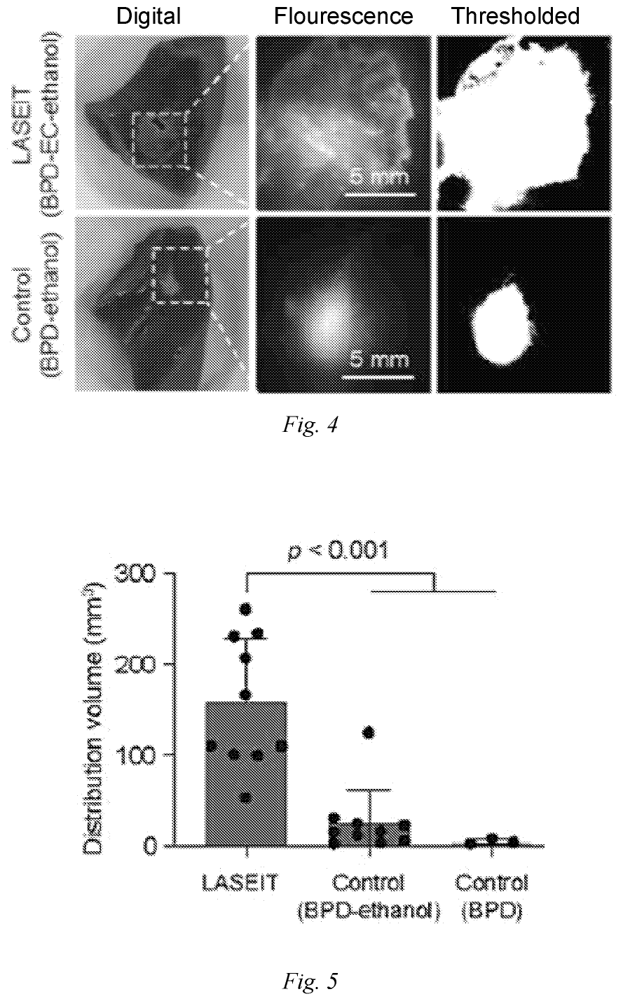

Similar to the evaluation of phantoms, fluorescence imaging of BPD was used to visualize and quantify the volume of LASEIT depot in swine liver tissues. The results are shown in FIG. 4 and FIG. 5. It was found that the combining of EC with BPD and ethanol beneficially increased depot volume by 6-fold, suggesting the importance of the interaction between BPD, EC, and ethanol in treating larger tumors.

### Punch Biopsies for Photochemical Characterization

For the tumor-mimicking mechanical phantom, 5 mm-deep portions of the phantom were removed via a biopsy tool with a diameter of 6.4 mm and placed into a 96-well, clear flat-bottom microplate. Singlet oxygen production was determined using Singlet Oxygen Sensor Green (SOSG) and a microplate reader before irradiation. The SOSG fluorescence was measured with excitation/emission wavelengths of 504/525 nm. The samples were then irradiated with a 690 nm continuous-wave laser at various radiant exposures, and singlet oxygen production was measured using SOSG and the microplate reader after irradiation. The difference between the obtained SOSG fluorescence values between pre- and post-irradiance samples constituted the relative singlet oxygen production of that particular punch biopsy sample.

Table 4 details a strategy for measuring the singlet oxygen production of a sample (e.g., phantom specimen from a punch biopsy). One replicate (a single injection of 300 μL of BPD-EC-Ethanol into a phantom) was provided to demonstrate how to estimate the relative singlet oxygen production of the BPD after light irradiance of the sample. 0 μM (EC-Ethanol only), phantom only, and BPD only (dissolved in DMSO) groups were included as negative controls, while the Rose Bengal group (type II photosensitizer capable of generating singlet oxygen via 525 nm light absorption) was included as a positive control. This strategy can be applied to estimate the relative production of singlet oxygen of photosensitizers in other liquid needle injections.

TABLE 4
Singlet oxygen production in a phantom.

| | Pre- | Post- | SO |
|---|---|---|---|
| | irradiation | irradiation | yield |
| Sample | RFU | RFU | (RFU) |
| 0 μM (EC-EtOH only) | 1403 | 1858 | 454 |
| Phantom only | 104 | 46 | −57 |
| 20 μM Rose Bengal | 18345 | 64703 | 46358 |
| 20 μM (BPD-EC-EtOH) | 5169 | 8663 | 3494 |

### BPD Stock Solution Preparation

To prepare the BPD stock solution, 5 mg of the BPD powder was dissolved with 1 mL of dimethyl sulfoxide in a 2 mL cryovial container. The molarity of the sample was confirmed with a microplate reader using the established molar extinction coefficient of BPD in dimethyl sulfoxide (DMSO; ˜34,895 M⁻¹ cm⁻¹ at 687 nm). The container was wrapped in aluminum foil and stored in a −20° C. freezer. The reconstituted solution was generally stable at −20° C. for several months but should only be at room temperature (25° C.) for a maximum of 4 hours.

### Singlet Oxygen Sensor Green (SOSG) Stock Solution Preparation

To prepare the SOSG stock solution, 100 μg of the SOSG powder was dissolved with 33 μL of methanol in the original container. The container was wrapped in aluminum foil and stored in a −20° C. freezer. The reconstituted solution was generally stable at −20° C. for several months but should only be at room temperature (25° C.) for a maximum of 4 hours.

### BPD-EC-Ethanol Solution Preparation

To prepare the BPD-EC-Ethanol solution, all actions were performed under as little ambient light as possible, or all solutions were kept wrapped in aluminum foil until needed. A vial of the BPD stock solution and SOSG stock solution preparation was thawed out at room temperature. Then, 0.9468 g EC was dissolved in 20 mL pure ethanol on a stirring hot plate at 300-400 rpm and at room temperature for 1 hour to form a 6% EC-Ethanol (w/v) stock solution. Next, 1500 μL of the 6% EC-Ethanol solution was transferred to a 2 mL microcentrifuge tube, and enough stock BPD solution was added to create the BPD-EC-Ethanol solution. For preparing solutions with different BPD concentrations, the volume of stock BPD solution could be changed as required. For example, with an 8 mM stock BPD solution, 3.75 μL of the stock BPD solution was added to 1494.75 μL of the 6% EC-Ethanol solution to create a 20 μM BPD-EC-Ethanol solution. Then, 1.5 μL of SOSG stock solution was added to the BPD-EC-Ethanol solution, resulting in a 5 μM SOSG solution in BPD-EC-Ethanol. The tube was inverted to mix the contents until the solution was homogenized. The BPD gave the solution a light-green color.

### Discussion

While ethanol ablation and photodynamic therapy have been individually studied and applied to cancers, the combinatorial therapy for such applications as described herein is the first of its kind. The inclusion of a photosensitizer (or any optical contrast agent) also provides an imaging modality to the drug formulation, which can prove useful for visualizing the distribution and depot volume within the tissues. The inclusion of EC in the formulation beneficially minimizes ethanol leakage in tumor models without any cytotoxic side effects. The data also suggest that the two techniques produce synergistic efficacy, providing greater target cell/tissue destruction than either technique alone.

The key steps of the protocol described herein include dissolving the BPD into the EC-Ethanol formulation, preferably taking care to minimize light exposure to the photosensitizer (or work in an environment with little to no blue or infrared light); injecting the formulation into the phantom and tissue samples with various injection protocols to determine how to optimize delivery; and characterizing the resulting distribution volumes via imaging and subsequent image analysis.

An important strength of this method lies in its inherent flexibility for accommodating different drug systems and environments to evaluate local delivery parameters. Substituting the provided example technologies for other liquid formulations described herein and using similar alternative imaging modalities would not require any major deviations in the methods and would achieve results that reveal distribution volumes and efficacy. Similarly, the methods described herein can be readily adapted to other tissue models by either modifying the agarose concentration of the phantoms or obtaining different animal tissues to inject.

The methods disclosed herein serve as a template to accommodate different tissue models and drug combinations to test various in vitro and ex vivo disease (particularly cancer) and other condition (e.g., lesions) treatments.

### Example 2. Gel Depot Evaluation

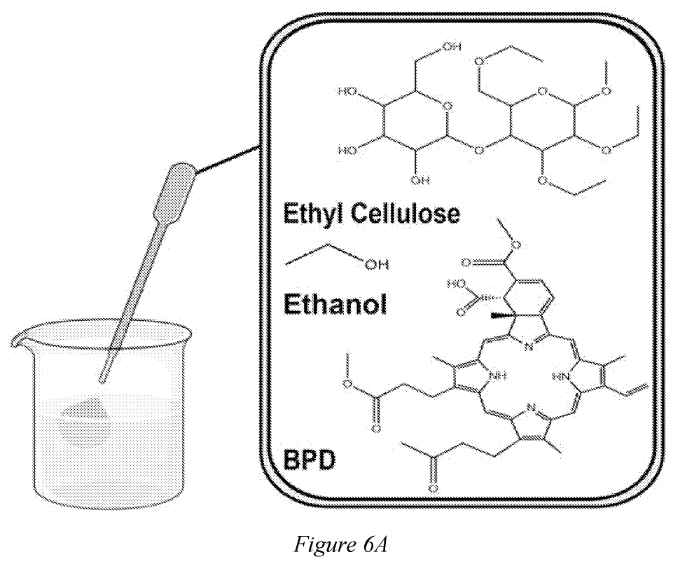

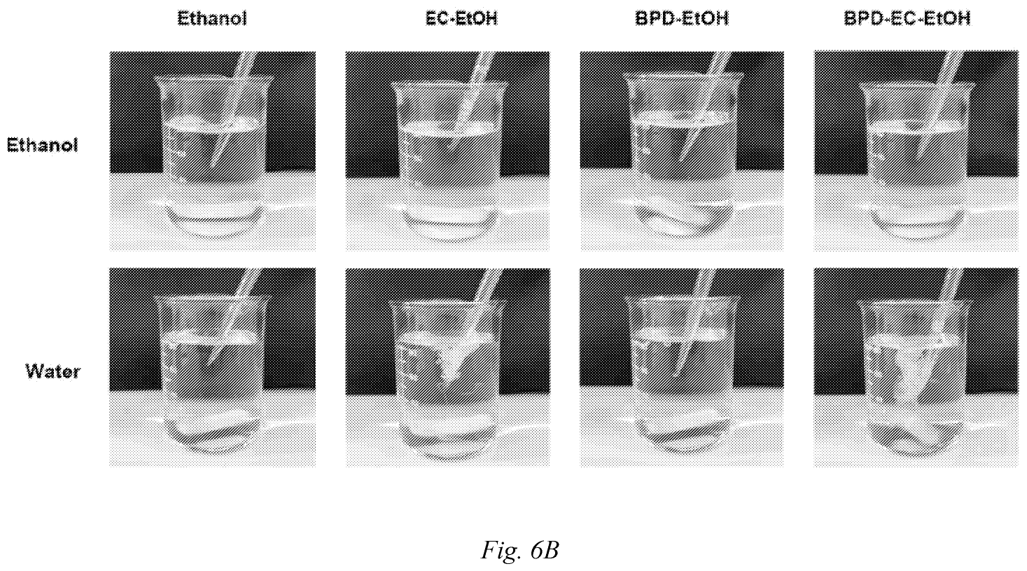

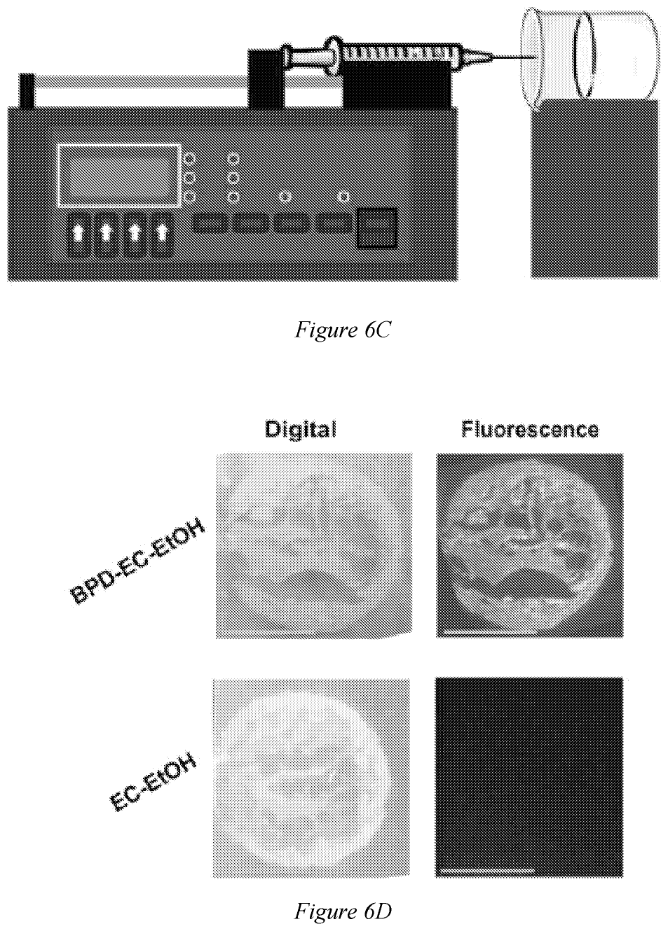

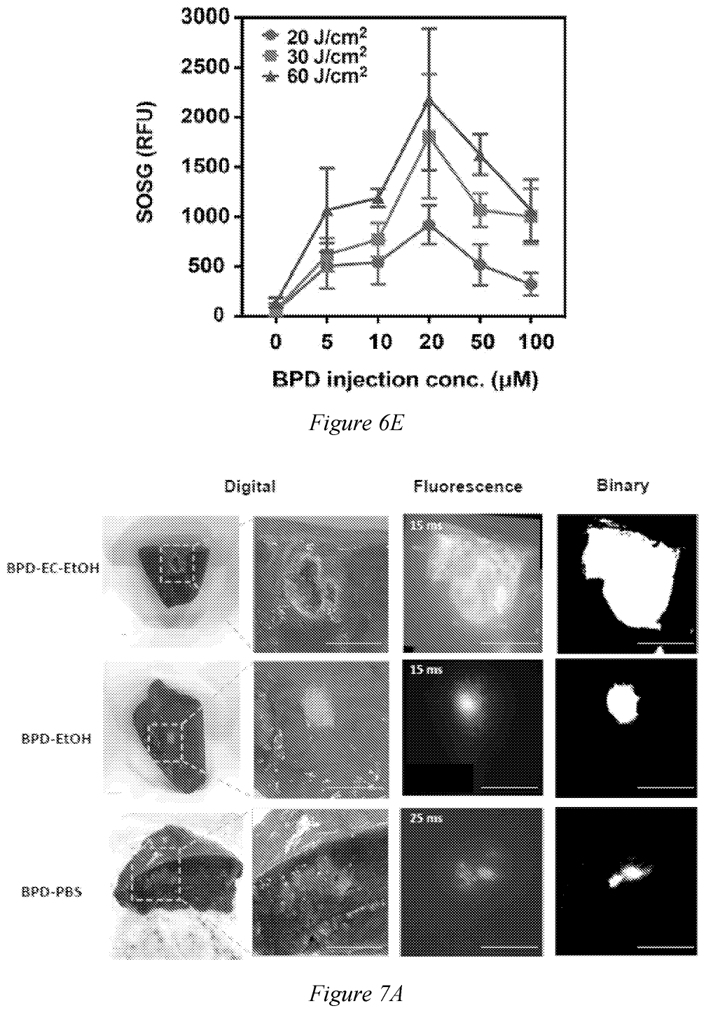

A soluble mixture of photosensitizer drug benzoporphyrin derivative (BPD), ethyl cellulose (EC) and ethanol (EtOH) was prepared using the protocols described in Example 1. This mixture was then used to form a gel depot/formation in water and in agar-based tissue mimicking phantoms. A schematic diagram and representative images of this mixture and depot in water are shown in FIG. 6A and FIG. 6B, while the experimental setup and representative images of formation in the phantom are shown in FIG. 6C and FIG. 6D.

Upon red light (690 nm) activation of BPD-EC-EtOH, the fluorescence signal generated from BPD can be used for imaging of the depot, as shown in FIG. 6E. Singlet oxygen sensor green (SOSG) assay revealed that BPD-EC-EtOH depot can be light (690 nm) activated to generate cytotoxic singlet oxygen molecules (¹O₂) in a BPD concentration and light dose-dependent manner.

This example shows that BPD-EC-EtOH beneficially forms a gel depot in aqueous medium or tissue phantoms, improving the retention of the BPD photosensitizer at the injection site. Further, the BPD-EC-EtOH gel depot can be activated by non-toxic red light (690 nm) for fluorescence imaging and singlet oxygen generation.

### Example 3. Gel Depot Formation and Fluorescence in Swine Liver Tissue

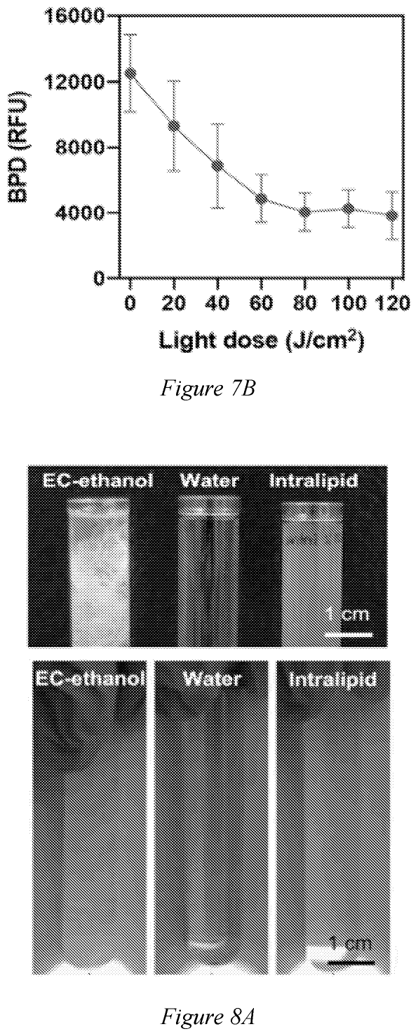

Compositions and depots were prepared and injected into swine liver tissue according to the protocols of Examples 1-2. Imaging was then conducted according to the procedures of Example 1. The results are shown in FIGS. 7A-7B. As shown in FIGS. 7A and 7B, BPD-EC-EtOH improves BPD retention in liver tissue, compared to using BPD-EtOH and BPD phosphate buffered saline (PBS) samples (scale bar=1 cm). In liver tissue, the singlet oxygen produced upon red light (690 nm) activation of BPD-EC-EtOH resulted in BPD photobleaching reduces BPD's fluorescence intensity. These data show that BPD-EC-EtOH can be injected into liver tissues to form a gel depot and improve BPD distribution, and further that BPD-EC-EtOH gel depot liver tissues can be readily activated by red light for fluorescence imaging and singlet oxygen generation.

### Example 4. Light Propagation

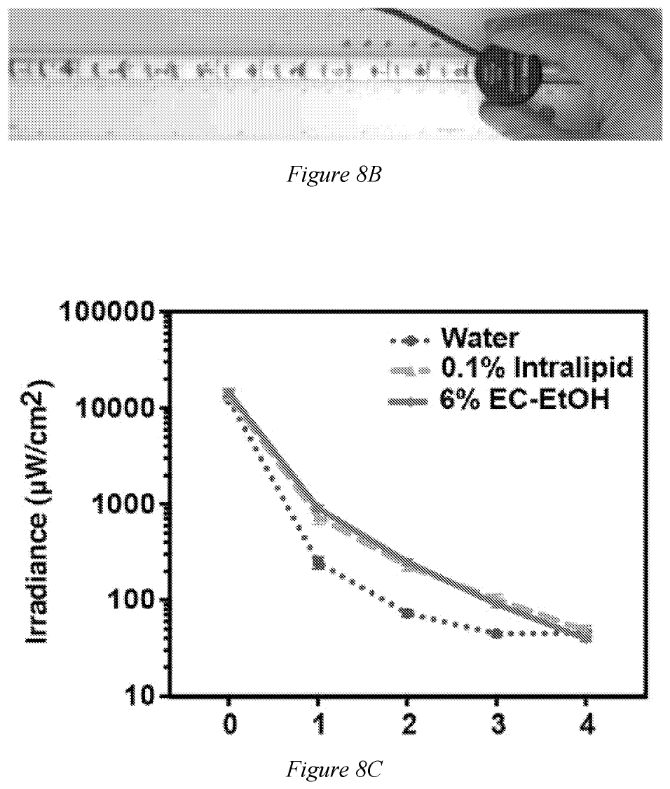

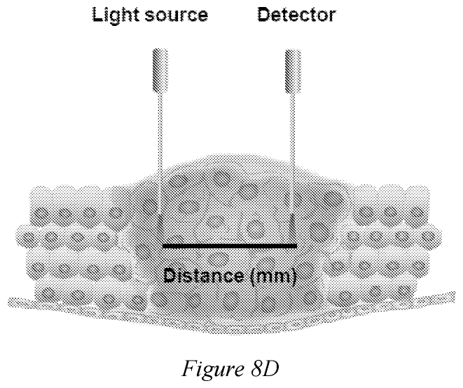

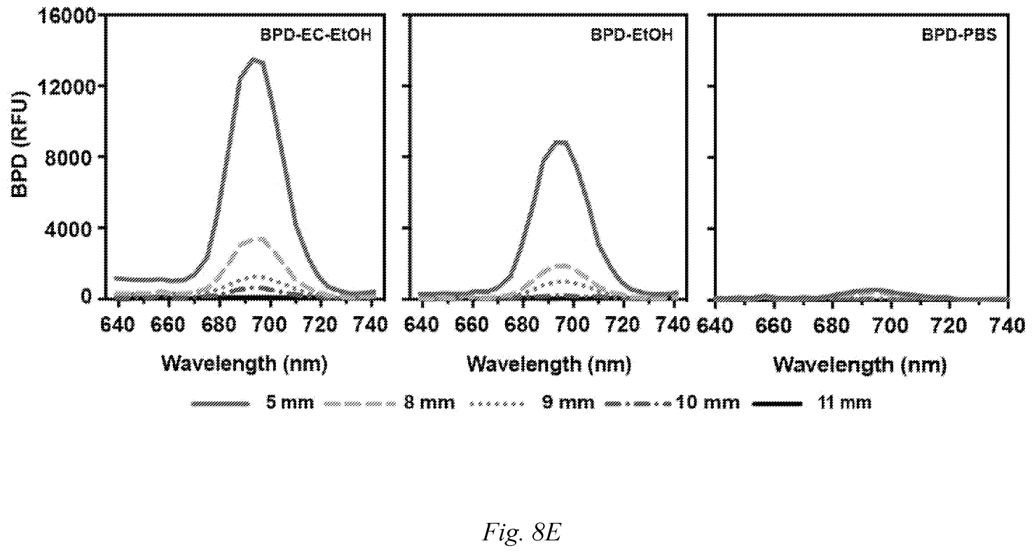

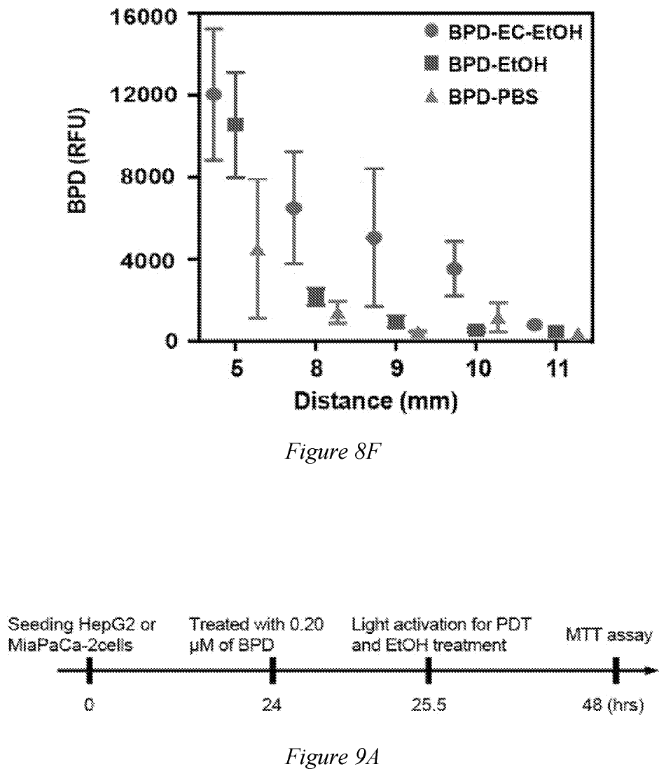

The ethyl cellulose (EC)-based gel depot described previously was evaluated for its ability to propagate light in test tubes, pipetts, and swine liver tissues. In particular, as shown in FIG. 8A, three samples, EC-based gel depot, water and 0.1% Intralipid® (a clinically used light scattering agent), were prepared test tubes and a light source (690 nm) was place at the bottom of each test tube for observation of light propagation. Both EC-based gel depot and Intralipid® improved light scattering in aqueous medium. As shown in FIG. 8B and FIG. 8C, light propagation in EC-based gel depot, water, or 0.1% Intralipid® was further quantified by measuring light irradiances (690 nm, 50 mW/cm²) at different distance (0-4 cm) from the light source. As shown in FIG. 8D, liver tissues injected with BPD-EC-EtOH, BPD-EtOH, or BPD-PBS were irradiated with a fixed 690 nm excitation source, and the BPD fluorescence spectra detected at different depth in the liver tissues. The results are depicted in FIG. 8E and FIG. 8F.

These data demonstrate that the ethyl cellulose (EC)-based gel depot improves the light propagation in test tubes, pipetts, and swine livers. Further, the results show that BPD-EC-EtOH depot can propagate red light in liver tissues up to 10 cm from the light source.

### Example 5. Synergistic Efficacy of BPD-Based Photodynamic Therapy & Ablation Methods

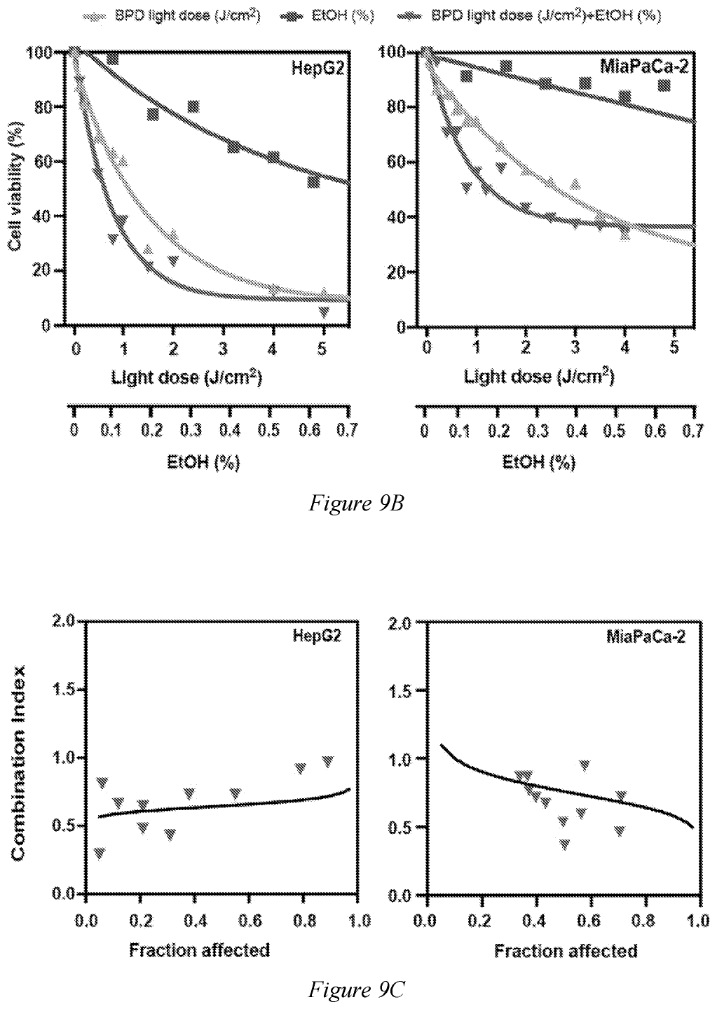

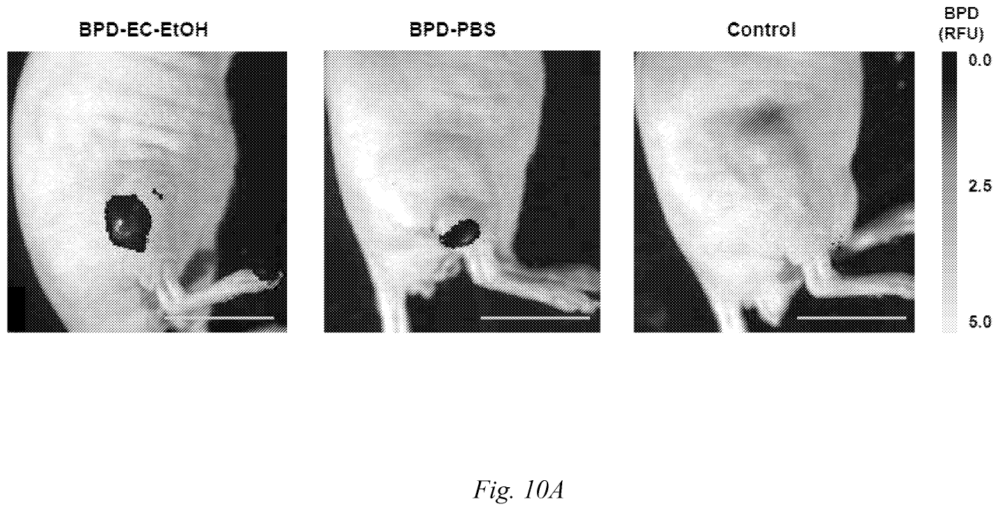

Although EC-ethanol treatment has tumor reduction capabilities, such treatments have resulted in 100% tumor regrowth 2 weeks after EC-ethanol ablation. Thus, there remains a need to develop a synergistic, combinatorial approach beyond simple EC-ethanol ablation. Accordingly, further evaluation of the efficacy of the compositions described herein was conducted to assess the synergistic effects of BPD-based photodynamic therapy in combination ethanol ablation in liver and pancreatic cancer cells. According to the protocols described in the examples and as shown in FIG. 9A, Liver cancer (HepG2) or pancreatic cancer (MIA PaCa-2) cells were seeded in 96 well plates at 20,000-25,000 cells/well and allowed to grow overnight. Cells were treated with 0.20 μM of BPD and/or EtOH (0-0.7%) for 24 hours prior to red light activation for photodynamic therapy (PDT). MTT cell viability assay was performed at 24 hours after PDT. The results are shown in FIG. 9B. Combination index (CI) was used to determine the degree of drug interaction, which defines synergism (CI <1), additive (CI=1) and antagonism effect (CI >1). The results are shown in FIG. 9C. These data show that the combination treatment of BPD-based photodynamiic therapy and ethanol ablation synergistically reduced liver and pancreatic cancer cell viability.

### Example 6 Pancreatic Tumor Mouse Model

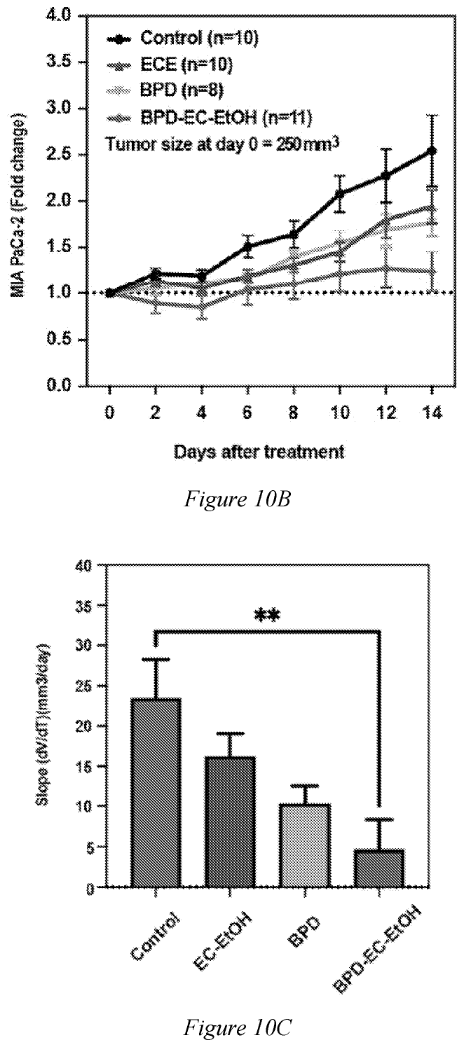

Mice bearing MIA PaCa-2 tumors (60 mm³ or 250 mm³) were divided into the following groups: (1) No treatment, (2) EC-EtOH, (3) BPD+hv (690 nm, 60 J/cm²), and (4) BPD-EC-EtOH+hv (690 nm, 60 J/cm²). Immediately after intratumoral injection of BPD-EC-EtOH (or controls), IVIS small animal imaging (reflected in FIG. 10A) shows the stronger BPD fluorescence signal from BPD-EC-EtOH, compared to BPD alone or no treatment. Fold changes in tumor volume were plotted as a function of time and are shown in FIG. 10B. Further, tumor growth rates were determined and are presented in FIG. 10C. Overall, BPD-EC-EtOH improved fluorescence imaging and treatment of pancreatic tumors in MIA PaCa-2 mice. IVIS imaging shows BPD-EC-EtOH depot improved BPD distribution in MIA PaCa-2 tumor in mice. Further, the combination treatment of photodynamic therapy and ethanol ablation can be achieved using BPD-EC-EtOH. Finally, the data show that light activation of BPD-EC-EtOH shows stronger anti-tumor efficacy, compared to light activation of BPD alone or EtOH ablation alone.

### Example 7. LASEIT Distribution

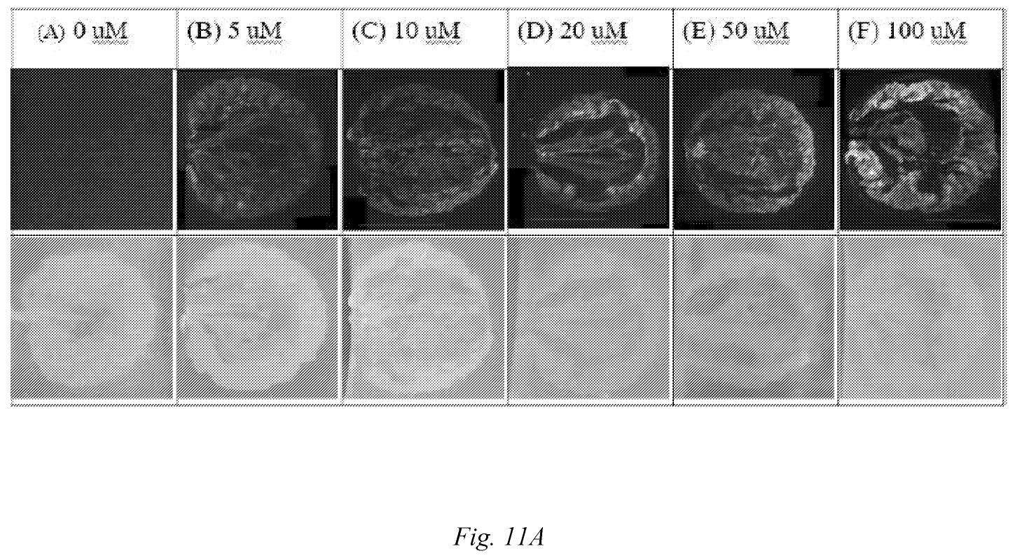

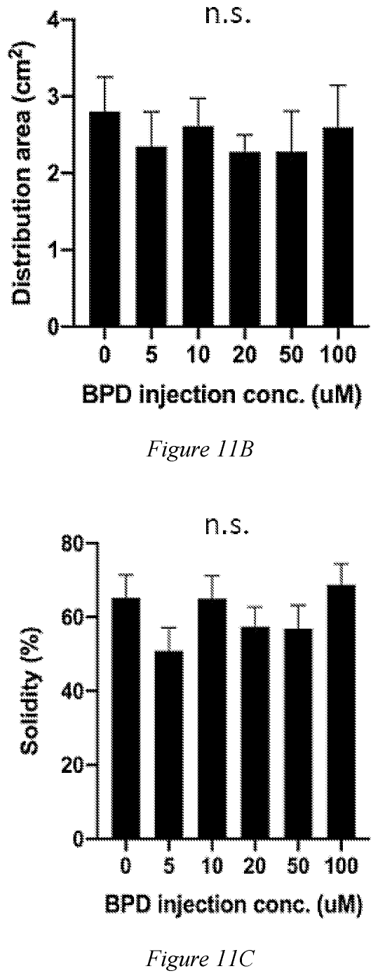

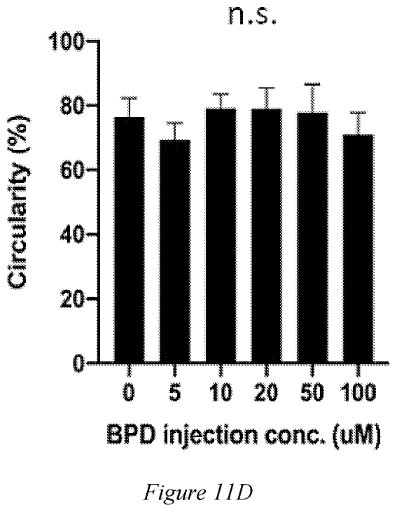

A panel of fluorescence and digital images of LASEIT injections of varying BPD concentrations in agar phantom substrates was generated following excitation with a 685 nm laser. The results are shown in FIG. 11A. As shown in this figure, the distribution zone of LASEIT in the phantoms is consistent across the BPD range. This is further evidenced by the distribution area, solidity and circularity, as shown in FIGS. 11B-11D, respectively. These data show that ethyl cellulose and BPD are well localized in the depot soon after the LASEIT injection in the phantoms. Further, the obtained distribution areas are consistent across BPD concentrations.

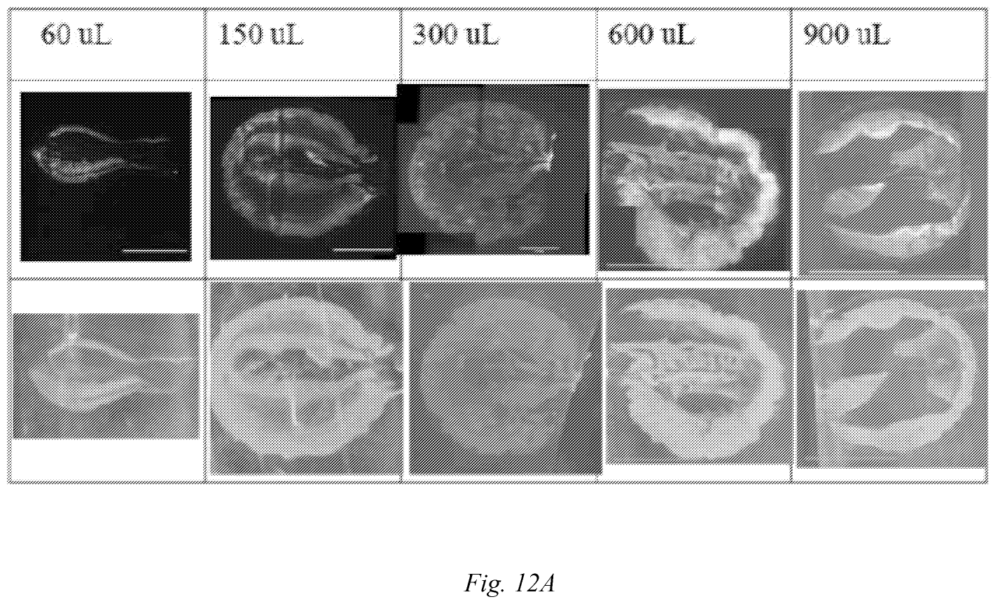

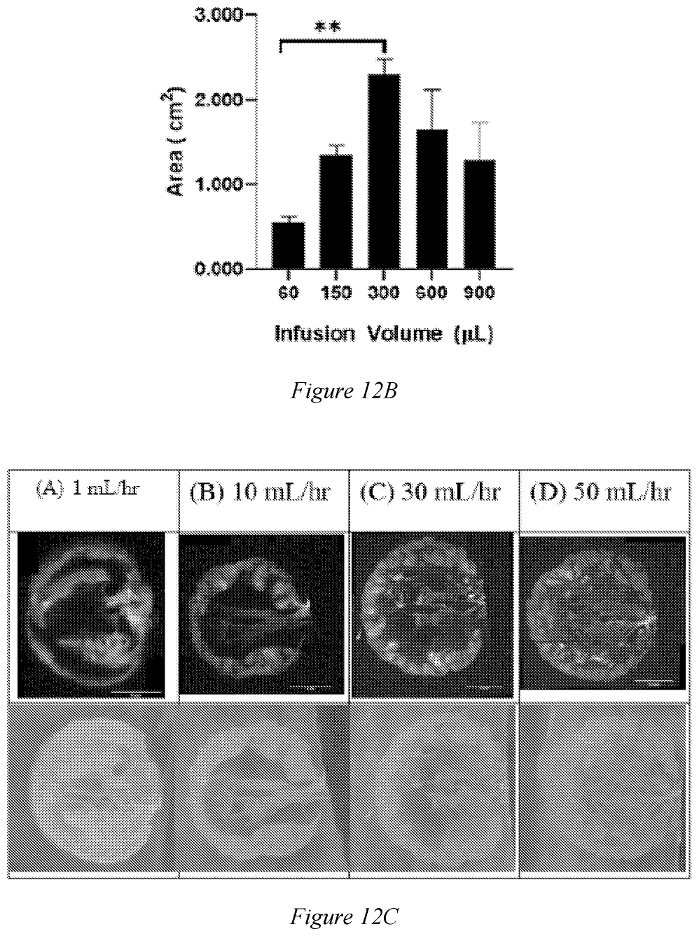

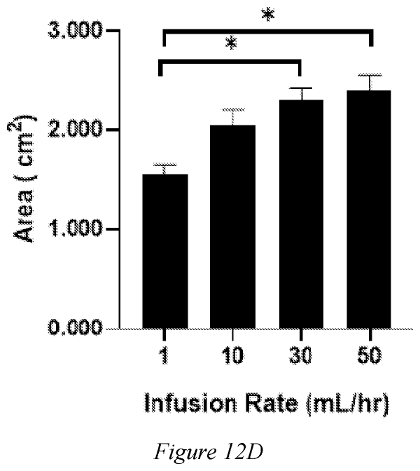

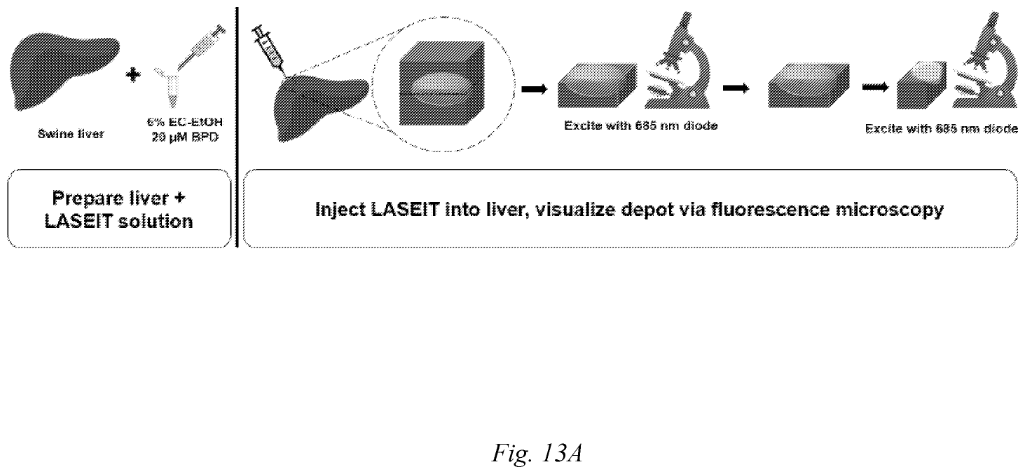

Next, LASEIT distribution area was evaluated as a function of infusion parameters. FIG. 12A shows a panel of representative fluorescence and digital images of LASEIT injections as a function of infusion volume, wherein scale bars=5 mm, except for the top row 600 μL and 900 μL images, where the scale bars=10 mm. FIG. 12B shows LASEIT distribution area as a function of infusion volume (n=5), where all error bars are SEM. (**: p<0.01). FIG. 12C shows a panel of representative fluorescence and digital images of LASEIT injections as a function of infusion rate, wherein the scale bars=5 mm. Finally, FIG. 12D shows LASEIT distribution area as a function of infusion rate (n=5), where all error bars are SEM (*: p<0.05). These figures show that generally, 300 μL maximizes distribution area in phantoms (as larger areas may have high backflow), meaning around this volume (about 100-300 μL) provides peak efficiency. Further, LASEIT infusion rates of greater than or equal to 30 mL/hr achieve peak distribution area in phantoms.

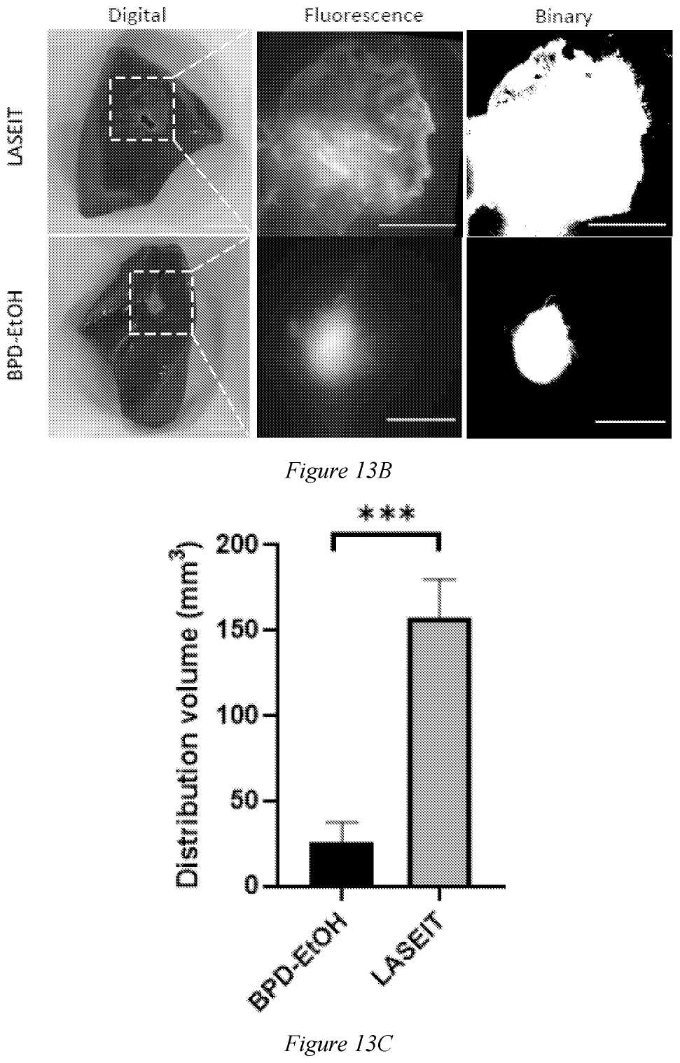

LASEIT distribution volume in excised swine liver was next assessed. The method of assessment, as depicted in FIG. 13A involved preparation of the liver and LASEIT solution. The LASEIT was injected into the liver to create a depot, and the depot was then visualized via fluorescence microscopy. The results were recorded in FIGS. 13B-13C, wherein error bars are standard error of the mean (SEM) (***: p<0.001). FIG. 13B shows representative digital fluorescence, and segmented binary images of both LASEIT (top row) and control (bottom row) in excited swine liver, wherein the bars for the digital images=10 mm and the scale bars for the other images=5 mm. FIG. 13C shows how LASEIT significantly increased the distribution volume in the tissues, compared to that for the BPD-EtOH. These data show that the presence of ethyl cellulose significantly increases volume in liver tissue and that without EC, there is very inefficient depot formation and low volumes. In particular, a lack of EC in the controls resulted in low distribution and volume retention for local delivery, which in turn suggests suboptimal tumor covers. In comparison, with EC, there is good depot formation and high volumes.

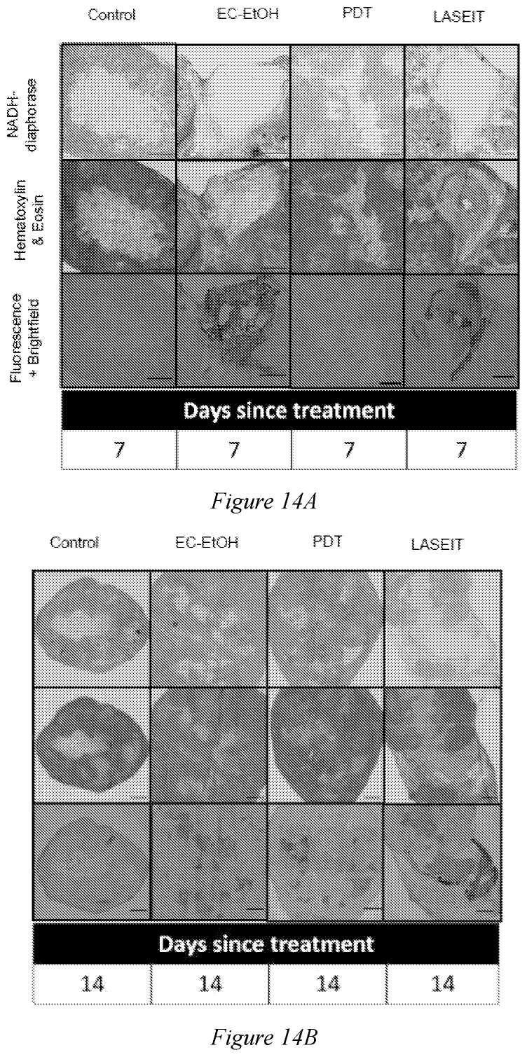

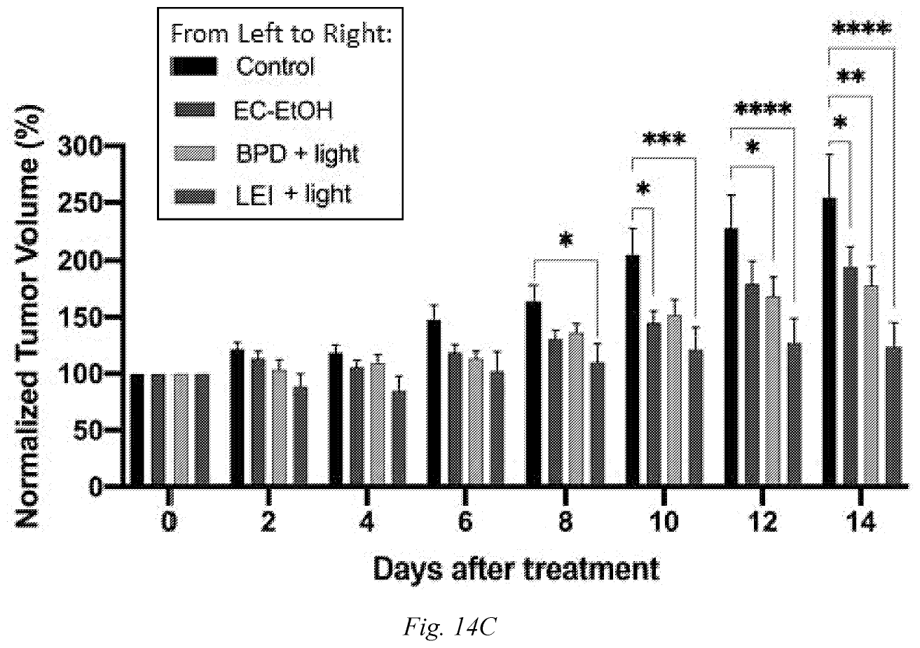

Finally, a histopathology assessment of MIA PaCa-2 tumors treated with LASEIT was conducted. Following LASEIT injection, the ablation zone within MIA PaCa-2 tumors was assessed. The results are shown in FIG. 14A and FIG. 14B. which contain representative digital images of unstained, NADH-diaphorase, and H&E tumor slides treated with LASEIT or its monotherapy counterparts, along with the tumor slides' representative fluorescence images (wherein black scale bars=1 mm). All tumors were collected 7 days (as shown in FIG. 14A) and 14 days (as shown in FIG. 14B) post-treatment. Further, FIG. 14C shows MIA PaCa-2 tumor size monitoring after treatment over the course of 14 days (n=8; error bars are SEM; significance values: *: p<0.05, **: p<0.01, ***: p<0.001, ****: p<0.0001). These data show that LASEIT retains BPD over 7 days, longer than the PDT treatment. Further, at 14 days, the LASEIT treatment displays the largest necrotic volume, compared to that of the monotherapies and the control. LASEIT treatment achieves superior reduction in tumor burden compared to the monotherapies.

### Example 8. Validation

Validation was done for the protocol of Example 1. To validate the ImageJ cylindrical volume measurement method, the accuracy and repeatability of the method were assessed through calculating the volume of a standardized flat cylindrical object (i.e., United States-minted pennies). Using the same fluorescence imaging setup, brightfield digital images of the coins were obtained in the same orientation as that for the phantom distribution volumes (i.e., the front and side cross-sections were imaged). After adding a scale bar and obtaining binary images, the front cross-section was traced via the Polygon tool, and the side cross-section was analyzed via the Local Thickness plugin. Six replicates were generated over three different coins.

To validate the novel MATLAB script (see Appendix) that calculates ellipsoidal volumes, the accuracy and repeatability of the code were assessed through calculating the volume of a standardized ellipsoidal object (i.e., Dayquil Liquicap gels). Using the same fluorescence imaging setup, brightfield digital images of the gels were obtained in the same orientation as that for the swine liver distribution volumes (i.e., the front and side cross-sections were imaged). After adding a scale bar and obtaining binary images, the MATLAB script was executed to obtain the volumes of the measured gel capsules. Six replicates were generated over two different capsules.

Table 5 displays the volume calculation of the coins, given the values of the three axes obtained from the MATLAB script. The sample acceptance criteria of ≤10% difference and ≤5% relative standard deviation (RSD) is also included, taking inspiration from ICH Q2(R1) guidelines. A percent difference of 3.9% and a percent RSD of 4.6% for six replicates was obtained, which passed the acceptance criteria and provided validation of the accuracy and repeatability of the ImageJ cylindrical volume calculations.

TABLE 5
Validation of the accuracy, repeatability tests of
the ImageJ volume calculation with the U.S. penny.

| | | | | | | | Expected | |
|---|---|---|---|---|---|---|---|---|
| | Area | Thickness | Vol. | Avg. | Std. | % | Volume | |
| Sample | (mm²) | (mm) | (mm³) | (mm³) | Dev. | RSD | (mm³) | \|% Difference\| |
| Penny1 | 286.4 | 1.445 | 413.9 | 416.5 | 19.0 | 4.6% | 433.2 | 3.9% |
| Penny2 | 282.9 | 1.396 | 395.0 | | | | | |
| Penny3 | 278.1 | 1.421 | 395.1 | | | | | |
| Penny4 | 282.2 | 1.568 | 442.5 | | | | | |
| Penny5 | 284.0 | 1.506 | 427.7 | | | | | |
| Penny6 | 283.1 | 1.501 | 424.9 | | | | | |

Table 6 displays the volume calculation of the Dayquil gels. The sample acceptance criteria of ≤10% difference and ≤5% relative standard deviation (RSD) is also included, taking inspiration from ICH Q2(R1) guidelines. A percent difference of 9.7% and a percent RSD of 1.9% for six replicates was obtained, which passed the acceptance criteria and provided validation of the accuracy and repeatability of the MATLAB ellipsoidal volume calculations.

TABLE 6
Validation of the accuracy and repeatability tests of the MATLAB script with the Dayquil gel.

| | | | | | | | | Expected | |
|---|---|---|---|---|---|---|---|---|---|
| | Axis A | Axis B | Height | Vol. | Avg. | Std. | % | Vol. | |
| Sample | (mm) | (mm) | (mm) | (mm³) | (mm³) | Dev. | RSD | (mm³) | \|% Difference\| |
| DQ1 | 21.1079 | 10.8597 | 10.2169 | 1223.2 | 1229.7 | 23.4 | 1.9% | 1121.1 | 9.7% |
| DQ2 | 21.4089 | 10.7135 | 10.1120 | 1211.4 | | | | | |
| DQ3 | 21.1328 | 10.8554 | 10.3988 | 1245.9 | | | | | |
| DQ4 | 21.3021 | 10.8565 | 9.9760 | 1205.0 | | | | | |
| DQ5 | 21.4586 | 10.9176 | 10.0103 | 1224.9 | | | | | |
| DQ6 | 21.3338 | 10.9349 | 10.4062 | 1267.9 | | | | | |

The experimental procedures used to evaluate and validate embodiments were developed in part using research in the field, for example, protocols described in the following references, each of which are incorporated by reference in their entirety: Shirley et al., Therapeutic endoscopic ultrasonography: intratumoral injection for pancreatic adenocarcinoma, Gastroenterol Res Pract. 2013 207129, (2013); Hong et al., Intratumoral Immunotherapy for Early-stage Solid Tumors, Clin Cancer Res. 26 (13), 3091-3099, (2020); Marabelle et al., Intratumoral immunotherapy: using the tumor as the remedy, Ann Oncol. 28 (suppl_12), xii33-xii43, (2017); Lencioni et al., Percutaneous ablation of hepatocellular carcinoma: state-of-the-art, Liver Transpl. 10 (2 Suppl 1), S91-97, (2004); Koda et al., Hepatic vascular and bile duct injury after ethanol injection therapy for hepatocellular carcinoma, Gastrointest Radiol. 17 (2), 167-169, (1992); Morhard et al., Development of enhanced ethanol ablation as an alternative to surgery in treatment of superficial solid tumors, Scientific Reports. 7 (1), 8750, (2017); Muñoz et al., Influence of injection technique, drug formulation, and tumor microenvironment on intratumoral immunotherapy delivery and efficacy, Journal for ImmunoTherapy of Cancer. 9 (2), e001800, (2021); Fujii et al., Optimal range of injection rates for a lymphatic drug delivery system, Journal of Biophotonics. 11 (8), e201700401, (2018); Morhard et al., Understanding Factors Governing Distribution Volume of Ethyl Cellulose-Ethanol to Optimize Ablative Therapy in the Liver, IEEE transactions on bio-medical engineering. 67 (8), 2337-2348, (2020); De Smet et al., Optimization of Drug Delivery Systems for Intraperitoneal Therapy to Extend the Residence Time of the Chemotherapeutic Agent, The Scientific World Journal. 2013 720858, (2013); Patsenker et al., Fluorescent Reporters for Drug Delivery Monitoring, Israel Journal of Chemistry. 60 (5-6), 504-518, (2020); Hellebust et al., Advances in molecular imaging: targeted optical contrast agents for cancer diagnostics, Nanomedicine (Lond). 7 (3), 429-445, (2012); Neefjes et al., Reporter gene comparison demonstrates interference of complex body fluids with secreted luciferase activity, Scientific Reports. 11 (1), 1359, (2021); Jun et al., Addressing the autofluorescence issue in deep tissue imaging by two-photon microscopy: the significance of far-red emitting dyes, Chemical science. 8 (11), 7696-7704, (2017); Baglo et al., Harnessing the Potential Synergistic Interplay Between Photosensitizer Dark Toxicity and Chemotherapy, Photochemistry, and photobiology. 96 (3), 636-645, (2020); Dodd et al., Minimally Invasive Treatment of Malignant Hepatic Tumors: At the Threshold of a Major Breakthrough, RadioGraphics. 20 (1), 9-27, (2000); Nief et al., Polymer-assisted intratumoral delivery of ethanol: Preclinical investigation of safety and efficacy in a murine breast cancer model, PLOS ONE. 16 (1), e0234535, (2021); N. M. Iniguez-Ariza et al., “Ethanol Ablation for the Treatment of Cystic and Predominantly Cystic Thyroid Nodules,” Mayo Clin Proc. 93 (8), 1009-1017, (2018); J. L. Mueller et al., “Optimizing ethyl cellulose-ethanol delivery towards enabling ablation of cervical dysplasia,” Scientific Reports. 11 (1), 16869, (2021); H. Zou et al., “Application of photodynamic therapy for liver malignancies,” Journal of gastrointestinal oncology. 11 (2), 431-442, (2020); C.-H. Ma et al., “Intratumoral Photosensitizer Delivery and Photodynamic Therapy,” Nano LIFE. 11 (02), 2130003, (2021); C. T. Inglut et al., “Systematic Evaluation of Light-Activatable Biohybrids for Anti-Glioma Photodynamic Therapy,” Journal of clinical medicine. 8 (9), 1269, (2019); B. J. Liang et al., “Breaking the selectivity-uptake trade-off of photoimmunoconjugates with nanoliposomal irinotecan for synergistic multi-tier cancer targeting,” Journal of Nanobiotechnology. 18 (1), 1, (2020); Y. Baglo et al., “Porphyrin-lipid assemblies and nanovesicles overcome ABC transporter-mediated photodynamic therapy resistance in cancer cells,” Cancer Lett. 457 110-118, (2019); J. Gavini et al., “Verteporfin-induced lysosomal compartment dysregulation potentiates the effect of sorafenib in hepatocellular carcinoma,” Cell Death & Disease. 10 (10), 749, (2019); A. Ntonas et al., “Comparative Anatomical Study Between the Human and Swine Liver and Its Importance in Xenotransplantation,” Cureus. 12 (7), e9411-e9411, (2020); S. Preibisch et al., “Globally optimal stitching of tiled 3D microscopic image acquisitions,” Bioinformatics. 25 (11), 1463-1465, (2009); N. Otsu, “A Threshold Selection Method from Gray-Level Histograms,” IEEE Transactions on Systems, Man, and Cybernetics. 9 (1), 62-66, (1979); and Guideline, I. H. T. Validation of Analytical Procedures: Text and Methodology. Q2 (R1). 1(20), 05, (2005).

The embodiments being thus described, it will be obvious that the same may be varied in many ways. Such variations are not to be regarded as a departure from the spirit and scope of the disclosure and all such modifications are intended to be included within the scope of the following claims.

APPENDIX: LiverDistributionVolume.m

```
%Quantifying the BPD-EC-Ethanol distribution volume in tissue platforms
close all; clear all;clc;
%% USER INPUT %%
%Ensure images are in the same folder as MATLAB script
img1 = imread(‘Sample_X_frontview’,‘JPG’); %front image name and file type
img2 = imread(‘Sample_X_sideview’,‘JPG’); % side image name and file type
folder = ‘C:\FolderLocation’; %specify location of files
imagename = ‘Sample_X’; %specify name of final processed images
scalebar = 5; %specify length of scale bar, in mm (horizontal orientation)
%%% END USER INPUT %%%
%% Calibrate image 1 scale bar
figure; imagesc(img1);
orient portrait
zoom on
pause( );
zoom off
[x1,y1] = (ginput(3));
%Specify ends of horizontal scale bar
scaleBar1_end1 = x1(1);
scaleBar1_end2 =x1(2);
scaleBar1 = abs(scaleBar1_end1 − scaleBar1_end2)/scalebar; %normalize length
%% Trace outline of image 1
figure, imshow(img1);
hold on;
h = imfreehand;
Lesion1 = createMask(h);
hold off;
%LesionMask
%If you didn't outline it perfectly, this gets rid of the little extra
%parts or bumps of the outline
CCtemp = bwconncomp(Lesion1);
d1 = regionprops(CCtemp,‘Area’);
celld1 = struct2cell(d1);
matd1 = cell2mat(celld1);
areaTH = matd1;
idx = find(areaTH<=100);
LesionMask1 = Lesion1;
for ttt = 1:1:length(idx)
  idx_temp = idx(ttt);
  LesionMask1(CCtemp.PixelIdxList{idx_temp}) = 0;
end
%This finds the center of the lesion and the boundaries
LesionMaskLabels = bwlabel(LesionMask1);
blobMeasurements1 = regionprops(LesionMaskLabels,‘Centroid’);
[B,L] = bwboundaries(LesionMask1);
%Plots the lesion
fontSize = 10;
labelShiftX = 35;
%This draws the boundaries and numbers each nucleus to the right of the
%centroid
img1d = im2double(img1);
figure; imshow(img1d);
hold on;
for k = 1:1
  boundary 1 = B{k};
  plot(boundary1(:,2), boundary1(:,1), ‘g’, ‘LineWidth’, 2)
  blobCentroid1 = blobMeasurements1(k).Centroid;
  %text(blobCentroid(1) + labelShiftX, blobCentroid(2), num2str(k), ‘Color’, ‘w’, ‘FontSize’,
fontSize, ‘FontWeight’, ‘Bold’);
end
hold off;
print (‘-djpeg’,‘-r300’,[folder ‘/’ imagename ‘_front’]);
%% Calibrate image 2 scale bar
figure; imagesc(img2);
orient portrait
zoom on
pause( );
zoom off
[x1,y1] = (ginput(3));
%Specify ends of horizontal scale bar
scaleBar2_end1 = x1(1);
scaleBar2_end2 =x1(2);
scaleBar2 = abs(scaleBar2_end1 − scaleBar2_end2)/scalebar;
%% Trace outline of image 2
figure, imshow(img2);
hold on;
h = imfreehand;
Lesion2 = createMask(h);
hold off;
%LesionMask
%If you didn't outline it perfectly, this gets rid of the little extra
%parts or bumps of the outline
CCtemp = bwconncomp(Lesion2);
d2 = regionprops(CCtemp,‘Area’);
celld2 = struct2cell(d2);
matd2 = cell2mat(celld2);
areaTH = matd2;
idx = find(areaTH<=100);
LesionMask2 = Lesion2;
for ttt = 1:1:length(idx)
  idx_temp = idx(ttt);
  LesionMask2(CCtemp.PixelIdxList{idx_temp}) = 0;
end
%This finds the center of the lesion and the boundaries
LesionMaskLabels = bwlabel(LesionMask2);
blobMeasurements2 = regionprops(LesionMaskLabels,‘Centroid’);
[B,L] = bwboundaries(LesionMask2);
%Plots the lesion
fontSize = 10;
labelShiftX = 35;
%This draws the boundaries and numbers each nucleus to the right of the
%centroid
img2d = im2double(img2);
figure; imshow(img2d);
hold on;
for k = 1:1
  boundary 1 = B{k};
  plot(boundary1(:,2), boundary1(:,1), ‘g’, ‘LineWidth’, 2)
  blobCentroid1 = blobMeasurements1(k).Centroid;
  %text(blobCentroid(1) + labelShiftX, blobCentroid(2), num2str(k), ‘Color’, ‘w’, ‘FontSize’,
fontSize, ‘FontWeight’, ‘Bold’);
end
hold off;
print (‘-djpeg’,‘-r300’,[folder ‘/’ imagename ‘_side’]);
%% Final calculations
LesionMaskLabels = bwlabel(LesionMask1);
CCTemp = bwconncomp(LesionMaskLabels);
%Calculates the major axis of each lesion
MajorAxisTemp = regionprops(CCTemp,‘MajorAxisLength’);
CellTemp = struct2cell(MajorAxisTemp);
MatTemp = cell2mat(CellTemp);
A = MatTemp/scaleBar1;
%Calculates the minor axis of each lesion
MinorAxisTemp = regionprops(CCTemp,‘MinorAxisLength’);
CellTemp = struct2cell(MinorAxisTemp);
MatTemp = cell2mat(CellTemp);
B = MatTemp/scaleBar1;
FrontArea = (A/2)*(B/2)*pi;
LesionMaskLabels = bwlabel(LesionMask2);
CCTemp = bwconncomp(LesionMaskLabels);
%Calculates the minor axis of each lesion
MinorAxisTemp = regionprops(CCTemp,‘MinorAxisLength’);
CellTemp = struct2cell(MinorAxisTemp);
MatTemp = cell2mat(CellTemp);
C= MatTemp/scaleBar2;
FinalVolume = 1.33*FrontArea*(C/2); %volume in mm\hat{ }3
MaximumDepth = A;
MinorAxis1 = B;
MinorAxis2 = C;
save([folder ‘/’ imagename],‘img1’,‘img2’,‘LesionMask1’,‘LesionMask2’,‘FinalVolume’,
‘MaximumDepth’, ‘scaleBar1’, ‘scaleBar2’);
```

1: A light-activatable, sustained-exposure composition comprising:
a solvent comprising an alcohol;
a cellulosic polymer; and
a photosensitizer.

2: The composition of claim 1, wherein the solvent comprises phenol, methanol, ethanol, absolute alcohol, isopropanol, propanol, butanol, isobutanol, glycerol, propyl iodide, lipiodol, glycerol, polidocanol, or a combination thereof.

3: The composition of claim 1, wherein the cellulosic polymer comprises ethyl cellulose (EC), methyl ethyl cellulose (MEC), carboxymethyl cellulose (CMC), carboxymethyl ethyl cellulose (CMEC), hydroxyethyl cellulose (HEC), hydroxypropyl cellulose (HPC), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), ethyl hydroxyethyl cellulose (EHEC), cyanothyl cellulose, alginate, chitosan, or a combination thereof.

4: The composition of claim 1, wherein the photosensitizer comprises a benzoporphyrin derivative, HPPH, WST-11, ALA, methyl-ALA, hexyl-ALA, chlorin e6 trisodium, mono-L-aspartyl chlorin e6, talaporfin sodium, redaporfin, temoporfin foscan, photofrin, phthalocyanine, or a combination thereof.

5: The composition of claim 1, further comprising an additional therapeutic agent.

6: The composition of claim 5, wherein the additional therapeutic agent comprises a chemotherapeutic agent, an immunotherapeutic agent, a gene therapy agent, or a combination thereof.

7: The composition of claim 6, wherein the chemotherapeutic agent comprises doxorubicin, paclitaxel, cisplatin, or a combination thereof; wherein the immunotherapeutic agent comprises a checkpoint inhibitor, monoclonal antibody, cytokine, or a combination thereof, or wherein the gene therapy agent comprises a plasmid, viral vector, RNAi molecule, or a combination thereof.

8: The composition of claim 1, wherein the photosensitizer is present in an amount of between about 5 M to about 100 μM.

9: The composition of claim 1, wherein the cellulosic polymer is present in an amount of 1-15% (w/v) relative to the total weight of the composition, and wherein the solvent is present in an amount of 85-99% (v/v) relative to the total volume of the composition.

10: The composition claim 1, wherein the composition comprises between about 1% (w/v) and 15% (w/v) cellulosic polymer-solvent.

11: A light-activatable, sustained-exposure composition produced by a process comprising:
dissolving a cellulosic polymer in a solvent comprising an alcohol to form a polymer-solvent solution;
dissolving a photosensitizer in the polymer-solvent solution to form the light-activatable, sustained-exposure composition; and
optionally adding an additional therapeutic agent to the light-activatable, sustained-exposure composition.

12: A method of treating a disease comprising:
administering to a target in need a therapeutically effective amount of a liquid, light activatable, sustained-exposure composition comprising a solvent comprising an alcohol;
a cellulosic polymer; a photosensitizer; and optionally an additional therapeutic agent; and
exposing the target to a light source thereby activating the photosensitizer.

13: The method of claim 12, further comprising any one or more steps of:
(a) step of monitoring the distribution of the composition in the target;
(b) ablating a portion or all of the target; or
(c) generating reactive oxygen species in an amount sufficient to induce necrosis or apoptosis in a portion or all of the target.

14: The method of claim 12, wherein the administering occurs by injection.

15: The method of claim 14, wherein the administering occurs at an injection rate of between about 10 mL/hour to about 100 mL/hour, at an injection volume of between about 100 μL to about 5000 μL, or at an insertion depth of between about 4 mm to about 120 mm.

16: The method of claim 12, wherein the light source reaches up to 1.5 cm in the target.

17: The method of claim 12, wherein the administering occurs between once a day to once a month over a period of three months to a year.

18: The method of claim 12, wherein the liquid composition becomes a gel upon the administering.

19: The method of claim 12, wherein the target is a tumor or a lesion.

20: The method of claim 12, wherein the disease is liver cancer, pancreatic cancer, cervical cancer, head and neck cancer, skin cancer, oral cancer, or breast cancer.
